(19) United States
(12) Patent Application Publication
(10) Pub. No.: US 2022/0202350 A1
(43) Pub. Date: Jun. 30, 2022

(54) MULTIPARAMETER NONINVASIVE SEPSIS MONITOR

(71) Applicant: Zynex Monitoring Solutions, Inc., Englewood, CO (US)

(72) Inventors: Thomas Sandgaard, Englewood, CO (US); Zachary Alan Kugler, Denver, CO (US)

(21) Appl. No.: 17/566,403

(22) Filed: Dec. 30, 2021

Related U.S. Application Data
(60) Provisional application No. 63/132,744, filed on Dec. 31, 2020.

(51) Int. Cl. A61B 5/00; A61B 5/0205; A61B 5/024; A61B 5/08; G16H 50/30

(52) U.S. Cl. CPC A61B 5/412; A61B 5/02055; A61B 5/02427; A61B 5/0816; A61B 5/742; A61B 2562/0271; A61B 5/7275; A61B 5/7221; A61B 5/6826; G16H 50/30; A61B 5/4842

(57) ABSTRACT

A system for monitoring sepsis comprises one or more sensors that noninvasively monitor a patient to produce sensor data and processing circuitry to: derive, from the sensor data, one or more physiological parameters of the patient that are indicative of a sepsis state of the patient; apply a set of rules to the one or more physiological parameters; determine a sepsis state for the patient based on the set of rules applied to the one or more physiological parameters; and output at least one signal indicative of the sepsis state of the patient. The system includes an output device that outputs a notification based on the at least one signal.

### CROSS REFERENCE TO RELATED APPLCIATIONS

This application claims the benefit of U.S. Provisional Application No. 63/132,744, filed on Dec. 31, 2020, and entitled “Multiparameter Noninvasive Sepsis Monitor”, which application is incorporated herein by reference in its entirety.

### BACKGROUND

Sepsis, the term for system-wide disfunction from infection, is developed in 1.7 million Americans each year and kills nearly 300,000. The prevalence of sepsis is partially due to the challenging nature of early diagnosis of sepsis, where delay in treatment quickly deteriorates a patient's prognosis.

### SUMMARY

Example aspects of the present disclosure include:

A system for monitoring sepsis, comprising: one or more sensors that noninvasively monitor a patient to produce sensor data and processing circuitry to: derive, from the sensor data, one or more physiological parameters of the patient that are indicative of a sepsis state of the patient; apply a set of rules to the one or more physiological parameters; determine a sepsis state for the patient based on the set of rules applied to the one or more physiological parameters; and output at least one signal indicative of the sepsis state of the patient. The system includes an output device that outputs a notification based on the at least one signal.

Any of the aspects herein, wherein the one or more sensors include a heart rate sensor, a temperature sensor, and a respiration rate sensor, and wherein the one or more physiological parameters of the patient include heart rate monitored by the heart rate sensor, temperature monitored by the temperature sensor, and respiration rate monitored by the respiration rate sensor.

Any of the aspects herein, wherein the output device includes a display that displays the notification to indicate the sepsis state of the patient.

Any of the aspects herein, wherein the processing circuitry applies the set of rules by comparing the one or more physiological parameters to one or more thresholds associated with the one or more physiological parameters, and wherein the processing circuitry determines the sepsis state for the patient based on the comparison.

Any of the aspects herein, wherein the one or more thresholds include a plurality of thresholds associated with possible sepsis states of the patient, wherein the possible sepsis states include a state of no sepsis, a state of potential onset of sepsis, a state of severe sepsis, and a state of septic shock.

Any of the aspects herein, wherein the plurality of thresholds include thresholds associated with nominal limits of the one or more physiological parameters, thresholds associated with abnormal limits of the one or more physiological parameters, and thresholds associated with critical limits of the one or more physiological parameters.

Any of the aspects herein, wherein the processing circuitry applies the set of rules by: comparing the one or more physiological parameters to one or more baseline values for the one or more physiological parameters, the one or more baseline values being specific to the patient; and determining a sepsis score based on the comparison, wherein the processing circuitry determines the sepsis state of the patient based on the sepsis score.

Any of the aspects herein, wherein the processing circuitry applies the set of rules by: determining a difference between a first physiological parameter of the one or more physiological parameters measured at a first time and the first physiological parameter measured at a second time; and determining an index value based on the difference, wherein the processing circuitry determines the sepsis state for the patient based on the index value.

Any of the aspects herein, wherein the processing circuitry determines the index value based on the difference by: multiplying the difference by a coefficient associated with the first physiological parameter to generate an impact value; and determining the index value by adding the impact value to a base index value, the base index value being determined by baseline values for the one or more physiological parameters specific to the patient.

Any of the aspects herein, wherein a value of the coefficient is based on a reliability of the first physiological parameter in predicting the sepsis state.

Any of the aspects herein, wherein coefficient varies as the reliability of the first physiological parameter in predicting the sepsis state changes.

A device for monitoring sepsis includes processing circuitry to: derive, from sensor data of one or more noninvasive sensors, one or more physiological parameters of a patient that are indicative of a sepsis state of the patient; apply a set of rules to the one or more physiological parameters; determine a sepsis state for the patient based on the set of rules applied to the one or more physiological parameters; and output at least one signal indicative of the sepsis state of the patient.

Any of the aspects herein, wherein the processing circuitry applies the set of rules by comparing the one or more physiological parameters to one or more thresholds associated with the one or more physiological parameters, and wherein the processing circuitry determines the sepsis state for the patient based on the comparison.

Any of the aspects herein, wherein the one or more thresholds include a plurality of thresholds associated with possible states of the sepsis state of the patient, wherein the possible states include a state of no sepsis, a state of potential onset of sepsis, a state of severe sepsis, and a state of septic shock.

Any of the aspects herein, wherein the plurality of thresholds include thresholds associated with nominal limits of the one or more physiological parameters, thresholds associated with abnormal limits of the one or more physiological parameters, and thresholds associated with critical limits of the one or more physiological parameters.

Any of the aspects herein, wherein the processing circuitry applies the set of rules by: comparing the one or more physiological parameters to one or more baseline values for the one or more physiological parameters, the one or more baseline values being specific to the patient; and determining a sepsis score based on the comparison, wherein the processing circuitry determines the sepsis state for the patient based on the sepsis score.

Any of the aspects herein, wherein the processing circuitry applies the set of rules by: determining a difference between a first physiological parameter of the one or more physiological parameters measured at a first time and the first physiological parameter measured at a second time; and determining an index value based on the difference, wherein the processing circuitry determines the sepsis state for the patient based on the index value.

Any of the aspects herein, wherein the processing circuitry determines the index value based on the difference by multiplying the difference by a coefficient associated with the first physiological parameter to generate an impact value; and determining the index value by adding the impact value to a base index value, the base index value being determined by baseline values for the one or more physiological parameters specific to the patient.

Any of the aspects herein, wherein a value of the coefficient is based on a reliability of the first physiological parameter in predicting the sepsis state.

Any of the aspects herein, wherein coefficient varies as the reliability of the first physiological parameter in predicting the sepsis state changes.

A method of electronically monitoring signals which are indicative of a patient condition to determine when to warn a caregiver of a condition which is indicative of sepsis in a patient, the method comprising: outputting a first signal from a noninvasive optical sensor applied to a digit of the patient, the first signal being indicative of an amount of light absorption by body tissue of the patient and of a heart rate of the patient; outputting a second signal from a noninvasive acoustic sensor applied to an area of skin around the patient's throat, the second signal being indicative of acoustically sensed tracheal sounds of the patient; outputting a third signal from a thermal sensor, the third signal being indicative of a temperature of the patient; processing the first signal to output first measurement values for the heart rate of the patient, processing the second signal to output second measurement values for a respiration rate of the patient, and processing the third signal to output third measurement values for the temperature of the patient; applying a plurality of predetermined rules to the first, second, and third measurement values; and outputting, based on the application of the rules, at least one of an audio or visual indication to said caregiver that sepsis does not likely exist for the patient, that sepsis potentially exists for the patient, or that sepsis likely exists for the patient.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

FIG. 1 is a general block diagram of a sepsis monitoring system according to at least one embodiment;

FIG. 2 is a general flow chart for a sepsis monitoring system according to at least one embodiment;

FIG. 3 illustrates an acoustic sensor for monitoring various physiological parameters of a patient according to at least one embodiment;

FIG. 4 illustrates additional sensors for monitoring various physiological parameters of a patient according to at least one embodiment;

FIG. 5 illustrates electrodes of an electrocardiograph for monitoring various physiological parameters of a patient according to at least one embodiment;

FIG. 6 illustrates electrodes of an electroencephalograph for monitoring various physiological parameters of a patient according to at least one embodiment;

FIG. 7 is an illustration of a multiparameter sepsis monitor interface according to at least one embodiment;

FIG. 8 is an illustration for indicating a patient's sepsis state according to at least one embodiment;

FIG. 9 is a flow diagram for a conditional sepsis state scoring algorithm according to at least one embodiment;

FIG. 10 illustrates a sepsis index graphic according to at least one embodiment;

FIG. 11 is a flow chart for determining a patient's sepsis index according to at least one embodiment;

FIG. 12 is an example measured temperature parameter for use in a sepsis index according to at least one embodiment;

FIG. 13 is an example measured respiration rate parameter for use in a sepsis index according to at least one embodiment;

FIG. 14 is an example sepsis index graphic according to at least one embodiment;

FIG. 15 is an example sepsis index graphic with a base index of 0 instead of 100 as in FIG. 14;

FIG. 16 illustrates a method according to at least one embodiment;

FIG. 17 illustrates a method according to at least one embodiment;

FIG. 18 illustrates a method according to at least one embodiment;

FIG. 19 illustrates an example implementation of a system according to at least one embodiment; and

FIG. 20 illustrates an example implementation of a system according to at least one embodiment.

### DETAILED DESCRIPTION

Sepsis in a patient may be defined as the systemic inflammation and dysregulation caused by an infectious source. Sepsis may cause life-threatening organ dysfunction due to a dysregulated host response to infection. The broad definition of sepsis includes an array of severities within the condition, generally presenting initially as systemic inflammatory response syndrome (SIRS), the systemic inflammatory process of the body in response to some perturbation in physiology. However, the cause of SIRS is not limited to infectious sources. Thus, for a patient's symptomatic presentation to be diagnosed as sepsis under currently accepted definitions, the presence of a source of infection (bacterial, fungal, viral, or parasitic) should be confirmed. Sepsis can quickly progress in severity to include organ dysfunction and hypotension, termed severe sepsis, and when the resulting hypotension cannot be remedied through standard fluid resuscitation methods, the condition has progressed into septic shock.

Despite the long-existing presence of sepsis in hospital settings, a solution to the medical problem of identifying sepsis and/or the onset of sepsis continues to elude professionals. Current methods for clinical diagnosis of sepsis suffer from complex protocols, need of frequent measurements of patient's vital signs and responsiveness, and invasive lab testing. Inventive concepts propose a noninvasive, multiparameter sepsis monitoring device that classifies and alerts caretakers to a patient's sepsis conditions and gives early warning of the onset of sepsis, reducing some of the burden of manual patient monitoring while improving prognosis of septic patients through proactive, early detection and alerting.

Annual hospitalizations for sepsis continues to increase without a corresponding increase in overall hospitalizations. The average length of stay for patients with sepsis may be longer than patients with other conditions not including sepsis, and the risk of mortality greater. This places an additional financial burden on hospitals and patients in addition to the increased risk of mortality.

Additionally, sepsis has been related to patient morbidity in survivors. Sepsis survivors show an increased incidence of three-year mortality, cognitive impairment, acute myocardial infarction (AMI) and functional disability.

Many sepsis cases onset outside of the hospital and organ dysfunction most closely related to mortality may present before clinicians recognize the presence of the infection. This is partly due to increasing reliance on laboratory tests to confirm presence of infection before initiating treatment of the infection; every hour of delay in treatment for sepsis increases the chance of mortality. The reliance on invasive, time-consuming, and reactive methods to confirm presence of infection contributes further to delay of treatment.

A comprehensive scoring system to improve early detection of sepsis by clinicians has been sought after for decades. However, the statistics of increasing incidence and mortality show that current methods are inadequate. This may be in part due to the observation that the current ranking and scoring systems for sepsis are based on largely retrospective analyses of patients who were confirmed to have a sepsis infection. Therefore, a need for real-time monitoring of physiological changes is prudent and necessary for predicting the onset of sepsis prior to the clinical diagnosis.

The signs and symptoms of sepsis are often subtle. The low survival rate of patients with severe sepsis indicates that current sepsis diagnostic strategies, tools, and methods are lacking. For example, conventional patient monitors give insufficient advanced warning of deteriorating patient health or the onset of serious conditions resulting from sepsis. A monitor that noninvasively measures patient condition to provide caregivers with a warning in advance of the onset of sepsis would be beneficial.

Many different physiological parameters have been implicated in the condition of sepsis, however as previously mentioned, some of the parameters require lab testing which introduces a delay in the timeline for treating patients while others are highly influenced by non-sepsis effects. Therefore, a beneficial method for monitoring patient condition, in regard to onset of sepsis, is through non-invasive monitoring in order to minimize delays in crucial time for detection and treatment. A non-invasive monitor according to inventive concepts that targets key physiological parameters may provide valuable “red flags” to clinicians which may speed up the initial assessment for those at risk of developing sepsis or progressing into severe sepsis or septic shock.

Non-invasively monitored parameters such as heart rate, body temperature, blood pressure, and respiratory rate, among others, have all been implicated as indicators of sepsis. Maintaining an adequate blood pressure has been implicated as one of the highest priorities in preventing progression from sepsis to septic shock, making it a reasonable choice for an early detection system. Body temperature is also a valuable early indicator for infection because it has been shown that temperature change is seen within 30-45 minutes of toxin presence in the blood.

A monitoring device which combines the indicative power of these parameters and others provides the opportunity for much earlier indication of the onset of sepsis in a patient. Utilizing a monitoring system which employs multiple noninvasive parameters, such as those mentioned above, among others, has the ability to decrease the time until alert, increasing positive outcomes for patients that are presenting onset of sepsis. Inventive concepts propose to accomplish this by providing real-time data on patient trending to provide early indication of sepsis and warning of a patient's decline. This is unique compared to the current ranking systems and other invasive monitoring modalities.

SIRS refers to criteria to identify the systemic activation of the body's immune response, such as from sepsis. SIRS classification manifests as the presence of more than one condition, for example, a temperature greater than 100.4° F. or less than 96.8° F.; a heart rate greater than 90 beats/min.; a respiration rate greater than 20 breaths/min; and/or blood test results demonstrating leukocytosis, leukopenia, or bandemia. Similar other sepsis identification and classification protocols exist, such as the newer QSOFA evaluation and SEPSIS-3 protocol. Thus, a sepsis monitor according to inventive concepts is responsive to more than one physiological parameter out of heart rate, respiration rate, temperature, blood pressure, and/or physiological parameters/other vital signs. Sourcing additional parameters that are indicative of sepsis onset, via manual or automatic input, such as from blood or other laboratory tests, or signals from other monitored physiological parameters, provides additional value to a sepsis monitor. While any single physiological parameter may be indicative of a patient's sepsis condition or provide advance warning of the onset of sepsis conditions, a patient monitor that uses multiple parameters, all which are indicative of sepsis conditions, allows for increased confidence and specificity for the detection and diagnosis of sepsis states and conditions.

In at least one embodiment according to inventive concepts, sepsis monitoring is based upon one or more physiological parameters, where each parameter has associated limits, trends, patterns, and/or variability definitions related to classifying sepsis or the onset of sepsis, independently or conjunctively. Physiological parameters being monitored may include, but are not limited to: Heart Rate, Respiration Rate, Blood Pressure, Temperature, Blood Analytes (e.g., Methemoglobin, Carboxyhemoglobin, Total Hemoglobin, Hematocrit, Deoxygenated Hemoglobin, Oxygenated Hemoglobin, BILIRUBIN), Electroencephalogram characteristics, Electrocardiogram, Bioelectrical Impedance, oxygen content (O2ct), perfusion index, and/or blood oxygen saturation (Sp02).

At least one example embodiment is directed to a patient monitoring method seeking to identify a sepsis condition in said patient. The method includes noninvasively generating a plurality of sensor signals (stored as corresponding sensor data) responsive to physiological parameters which are indicative of a sepsis condition or the impending onset of sepsis conditions in a patient, where the measurement of multiple non-invasive parameters provides for higher sensitivity to, and specificity of, patient sepsis conditions beyond that of single-parameter monitoring apparatuses/methods. The method includes electronically computing measurements for said physiological parameters from various sensor signals, with said parameters including at least heart rate, temperature, and respiration rate. The method includes said respiration rate parameter responsive to measurements taken by an acoustic sensor applied to the neck, or through an optical sensor on the skin surface, and said heart rate parameter responsive to measurements taken from an electrocardiographic signal or by a pulse oximeter or other optical sensor applied to a digit or other part of the body. The method includes applying a plurality of predetermined rules and/or conditions to the physiological parameters so as to determine the onset of sepsis. Determining the onset of sepsis may include using a composite sepsis index to represent the state of sepsis conditions. The method includes indicating to an observer the potential existence of the sepsis condition for a patient.

At least one example embodiment is directed to a method of electronically monitoring signals which are indicative of a patient condition to determine when to warn a caregiver of a patient's condition(s) that is indicative of sepsis. The method includes outputting a first signal from a noninvasive optical sensor applied to a digit indicative of an absorption of light by body tissue of the patient, where said first signal also indicative of one or more physiological parameters of said patient, including heart rate. The method includes outputting a second signal from a noninvasive acoustic sensor applied to an area of skin near a patient's throat or chest, where said second signal is indicative of acoustically sensed tracheal sounds or using a signal output from the same aforementioned optical sensor applied to a digit, or from a different optical sensor. The method includes outputting a third signal from a thermal sensor wherein the signal is indicative of a temperature of the patient. The method also includes the possibility of outputting additional signals from the same aforementioned or differing sensors wherein the signal is indicative of another parameter of the patient that can be indicative of sepsis or the onset of sepsis. The method includes electronically processing said first signal to output measurement values for said one or more physiological parameters including at least the heart rate, processing said second signal to output measurement values for respiration, processing with said processor said third single to output measurement values for said temperature, and processing with said additional signals to output values for additional parameters of interest in sepsis diagnostics. The method includes electronically applying with said processor a plurality of predetermined rules and conditions to said measurement values, and when said application of said rules and conditions indicates said patient condition is indicative that sepsis potentially exists, or that sepsis likely exists, outputting at least one of an audio or visual indication to said caregiver that sepsis likely does not exist, or that sepsis potentially exists, or that sepsis likely exists, respectively according to the indication from the application of the rules and conditions.

An aspect of a sepsis monitor may include various sensors, interfaces, mechanisms, and processes used to measure and quantify physiological indicators of sepsis presenting in a patient and/or the warning signs presenting of the oncoming onset of sepsis. In one embodiment, acquisition of a patient's physiological parameters that are indicative of sepsis or the onset of sepsis is performed by the sepsis monitoring device. Signals for each parameter may be sampled from a physiological signal and analyzed to derive the present value of the physiological parameter or vital sign value of interest. Rules, criteria, and transforms are applied to the physiological parameters to determine the onset of the sepsis condition which is displayed and indicated to a user.

Aspects of a sepsis monitor include the specific parameters and vital signs being measured from a patient and the unique sensitivities of each parameter to the state of sepsis or onset of sepsis. A set of processing instructions that extracts indications of from each measured signal for a given parameter provides specified mechanisms for sampling, conditioning, filtering, or any other applicable process for extracting features applicable to the detection of sepsis or the onset of sepsis. Secondary layers of processing instructions combine extracted features, patterns, or variables of value across multiple parameters where applicable to further refine features into classifying components for determination of sepsis or onset of sepsis in a patient.

An embodiment of a sepsis monitor that utilizes heart rate as a vital sign that is indicative of sepsis or the onset of sepsis measures the patient heart rate from one or more sources including, but not limited to the following: an electrocardiographic signal measured by an electric potential signal picked up through two or more electrodes on the patient; a photoplethysmograph (PPG) signal picked up optically from one or more locations on the patient's body; and/or a bioelectrical impedance signal measured across the chest of a patient.

At least one embodiment of a sepsis monitor utilizes blood pressure (or other mechanical output indicators of cardiac performance) as a vital sign that is indicative of sepsis or the onset of sepsis and may measure blood pressure from one or more sources including, but not limited to the following: a photoplethysmograph signal picked up optically from one or more locations on the patient's body; a noninvasive blood pressure (NIBP) derived from a blood pressure sensor, such as an automatically controlled inflatable cuff and corresponding acoustic sensor (i.e. a continuous NIBP (CNIBP) measurement device or an intelligent cuff inflation (ICI) device).

At least one embodiment of a sepsis monitor utilizes respiration rate (or other mechanical output indicators of pulmonary performance) as a vital sign that is indicative of sepsis or the onset of sepsis. The respiration rate may be measured by one or more sources including, but not limited to the following: a photoplethysmograph signal picked up optically from a variety of locations on the patient's body; a noninvasive acoustic sensor placed on the patient's body to pick up acoustic signals corresponding to the breathing cadence and/or depth of the patient; a capnograph signal via a patient's airway, which may also produce indicators of gas exchange, concentration, and partial pressures.

An embodiment of a sepsis monitor that utilizes temperature (skin surface or core body) as a vital sign that is indicative of sepsis or the onset of sepsis measures patient temperature from sources including, but not limited to: a signal picked up optically from one or more locations on the patient's body, such as from infrared emission off of the surface of the skin; a noninvasive thermocouple or thermistor placed on the patient's body to generate an electrical potential proportional to the measured temperature.

An embodiment of a sepsis monitor that utilizes various blood analytes (e.g., Methemoglobin, Carboxyhemoglobin, Total Hemoglobin, Hematocrit, Deoxygenated Hemoglobin, Oxygenated Hemoglobin, Bilirubin, Oxygen Content (O₂ct) and Saturation Percent of Oxygen (SpO₂), and/or perfusion index as detected by a pulse oximeter) as vital signs that are indicative of sepsis or the onset of sepsis may noninvasively measure the patient from one (or multiple) tuned set(s) of a Light Emitting Diode(s) (LEDs) or laser diode(s) with a corresponding photodiode(s) where the wavelength emission and receptor sensitivity is based on absorbance of the blood analyte(s) being measured. This set of optical emitters/receivers can be placed on the body in similar locations for optimal photoplethysmography measurements on the body.

An embodiment of a sepsis monitor that utilizes electroencephalogram measurements as a vital sign that is indicative of sepsis or the onset of sepsis may measure the patient from typical electrode usage and placement for other electroencephalograph signal acquisition from the body. An electroencephalogram signal provides the diagnostic ability for detection of septic encephalopathy, a condition that may appear in patients diagnosed with sepsis, making it a highly specific parameter for indication of sepsis or the onset of sepsis.

In other embodiments, parameters and vital signs that are indicative of sepsis or the onset of sepsis are measured non-invasively on the patient by the sepsis monitor, however, some of these parameters, along with many others, can be measured through invasive methods (i.e., lab work, blood draws, imaging, invasive procedures, etc.). Parameters that are indicative of sepsis or the onset of sepsis, by which the sepsis monitor does not directly acquire the signals for the parameter from the patient, can be indirectly input into the monitor either through manual user-interfaces, automatically through an Electronic Health Record (EHR) or Electronic Medical Record (EMR), through a digital communication interface or network between systems, or other automatic or manual interfacing method to allow the sepsis monitor access to the parameter or vital sign indication. Additionally, a sepsis monitor may prompt a clinical caretaker, when the patient under monitoring presents sepsis conditions or the onset of sepsis, to obtain additional metrics or measurements that would be pertinent for classification and/or diagnosis of sepsis or the onset of sepsis; for example, a sepsis monitor that uses non-invasive measurement apparatuses and techniques may not be capable of obtaining a metric such as the White Blood Cell count in a patient—the sepsis monitor could prompt a clinician to obtain that measurement through a lab test and input the value manually into the sepsis monitor (or have it automatically parsed from the patient's EMR) to use as a parameter in the detection of sepsis or the onset of sepsis.

In one embodiment, a sepsis monitor measures a patient's vital signs and physiological parameters that are indicative of sepsis and the onset of sepsis. While individual parameters may be indications of sepsis or onset of sepsis, a sepsis monitor will measure a selection of multiple parameters that are all indicative of sepsis or the onset of sepsis. The indications of parameters for sepsis are combined to attribute a higher confidence in the diagnosis of sepsis and/or the onset of sepsis in a patient. The indications of sepsis for individual parameters can be combined into a singular indication for a clinical end-user to make a diagnosis on the state of sepsis and/or the onset of sepsis for a patient being monitored. In this principal embodiment, patient monitoring begins in a known state where there is no presence of sepsis, such as immediately after a clinical operation, after blood tests to verify a lack of infection, etc. For a given patient, the monitor determines the non-septic baselines for each individual parameter in the group of parameters being monitored. Individual parameters may change from the baseline non-septic range; these changes could indicate the onset or state of sepsis in a patient. Parameters' changes are converted into a percent change values (compared to starting baseline values). Physiological parameters being monitored may not be of equal significance as indicators for sepsis or the onset of sepsis; each parameter will have a multiplier value (a static constant value or variable value) that is used to properly weight the significance of a parameter in determining the onset of sepsis or septic state of a patient. Each parameter's multiplier value and percent change value are multiplied together and the resulting value for the plurality of the parameters are summed together, creating a singular indicative value representing the septic state or onset of sepsis in a patient, i.e., a sepsis index.

Similar to tracking changes in baseline values, at least one embodiment may track changes in individual parameters and classify their indication of sepsis by their adherence to thresholds for a patient's septic classification or onset of sepsis. Where the current measured value of a parameter resides in the scale of thresholds determines its value in a singular sepsis value. This parameter's resulting value is summed with the same resulting value for the plurality of the parameters, creating a singular indicative value representing the septic state or onset of sepsis in a patient, i.e., a sepsis score (point-based). Additionally, a rule-based criteria system could be applied to individual parameters or sets of parameters to also create point values that are summed into a sepsis score; for example, if blood pressure is stable and nominal for a period of time and then drops quickly and is sustained, a point may be added to the sepsis index, creating a greater indication of sepsis for the clinical user to interpret from the sepsis index value.

An aspect of a sepsis monitor is the classification and reporting of a patient's sepsis conditions to a clinical caretaker. Using a plurality of a subset of physiological parameters that are indicative of septic status or onset of sepsis, aforementioned embodiments of a sepsis monitor use either a combinatory ‘Sepsis Index’ value or a state-determinate ‘Sepsis Score’ value with contributing sequences, patterns, or thresholds that go into a singular indication of a patient's sepsis score. An aspect of a reliable sepsis monitor uses a combination of these methods to present a septic status of a patient to the clinical caretaker. Additional methods of using machine learning to extract principal components or patterns from a plurality of physiological parameters based on outcomes of septic onset in monitored patients to further improve the sensitivity and specificity of individual or combined parameters in detection of features that are indicative of sepsis or the onset of sepsis in a patient.

A sepsis monitor for patient diagnostic purposes includes mechanisms by which the diagnostic information is conveyed to the clinical end-user(s). Sepsis can vary in patients by presentation of symptoms, progression and onset times, severity, and prognosis. Given this variability, an embodiment of a sepsis monitor shows an enumerated categorization of sepsis in the patient being monitored. For example, a sepsis monitor may display the distinct state of sepsis in a patient out of the following: no sepsis, onset of sepsis, sepsis, septic shock. In a variation of this, a sepsis monitor embodiment shows a sepsis index or score value that aids clinical end-users in either the diagnosis of septic state in a patient or by showing the progression of the patient's septic state as a progression of an index or score value over time. Additionally, these values can be directly linked to the enumerated states of sepsis in a patient as aforementioned. A sepsis monitor embodiment uses a combination of these mechanisms for conveying the septic status of a patient to the clinical end-user.

An important aspect for the use of a sepsis monitor includes a potential interface with a therapy delivery device to create a closed-loop feedback detection and administration solution for sepsis treatment. For example, a sepsis index (or other sepsis indicative metric produced by the monitor) may have a baseline value of 100 for an indication of no sepsis based on the patient's baseline non-septic parameter values. As the sepsis index value begins decreasing, an electronically administered therapy (i.e., antibiotics for an infection, fluid administration to maintain blood pressure, etc.) can be configured (such as an infusion pump) with the aim to improve patient prognosis. The therapy may respond to the sepsis index value in a proportional manner where a controlled feedback loop between the device produces the effect of increasing the therapy as a patient's septic condition declines. As the sepsis index returns towards 100, signifying patient's septic condition improving, the therapy can be reduced. Multiple therapies can be interlinked with the sepsis monitor with this method, such as a fluid volume monitor along the sepsis monitor and administration devices for antibiotics, vasopressors, and fluids.

Common challenges of real-time continuous patient monitoring include portability, connectivity, patient interfacing and clinical restrictions (size, body location(s), connection(s), disposability, among others. An aspect of any patient monitor is how the monitor interfaces with the patient. A basic embodiment features a monitor with various peripheral sensors that attach to the patient in the corresponding locations necessary for monitoring. Another embodiment features a patient-wearable device that has monitoring and interface components built into the wearable, reducing size, increasing portability, and allows for more mobility. Additionally, this wearable is physically or wirelessly connected to a monitor/display unit. Another embodiment features a wearable-only sepsis monitor that is outfitted with appropriate networking interfaces (i.e., Bluetooth, Wi-Fi, Cellular Data) allowing for a patient to be monitored remotely through the internet, either in a clinical environment or from home. The wearable sepsis monitor transmits parameter and sepsis data through the internet to a clinical caretaker—the wearable connects directly connect to the internet or, using an intermediate communication technology like Bluetooth or Wi-Fi, use the patient's home internet connection or mobile phone/device to transmit the information to a caretaker.

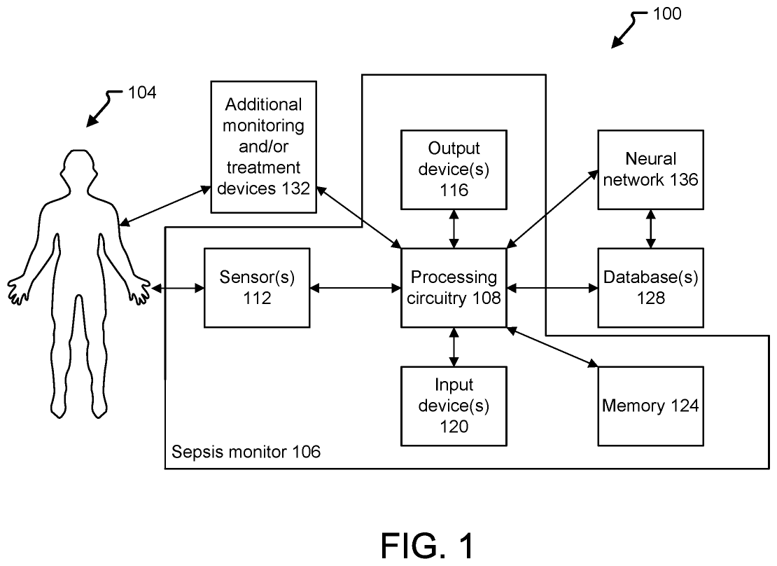

FIG. 1 illustrates a system 100 for monitoring sepsis in a patient 104 with a sepsis monitor 106. As shown, the system 100 may include the sepsis monitor 106 with processing circuitry 108, one or more sensors 112, one or more output devices 116, one or more input devices 120, and memory 124. The system 100 may further include one or more databases 128, one or more additional devices 132 (e.g., additional patient monitoring devices and/or treatment devices), and a neural network 136.

As described in more detail below, the sepsis monitor 106 may be a single instrument incorporating various hardware, circuits, and/or software/firmware for noninvasively generating sensor signals or sensor data, deriving physiological parameters from the sensor signals, and processing those parameters to generate the indicators and controls for a sepsis state of a patient as described herein. Additionally, the sepsis monitor 106 may integrate one or more standalone instruments or plug-ins, each of which measure particular physiological parameters and/or produce specific sensor signals for deriving particular physiological parameters. These plug-in units may include blood analyte and parameter monitors (e.g., a pulse oximeter), respiration rate monitors, blood pressure monitors, ECG monitors, PPG monitors, EEG monitors, and capnometers, to name a few non-limiting examples. Here, it should be appreciated that the sepsis monitor 106 may also include the additional devices 132 in that one or more of the additional devices 132 may be incorporated into the same device as the sepsis monitor.

In an embodiment, the sepsis monitor 106 includes input and output peripherals (I/O) which can extend user interactions and enable communications with other devices (e.g., devices that treat sepsis or other monitoring devices), patient records and databases, and the internet, to name a few I/O usage examples.

A sepsis monitor 106 may include pre-processing functionality, metric analysis functionality, post-processing functionality, and/or a device/peripherals controller. Sepsis monitor pre-processing components may receive inputs including real-time physiological parameter measurements, historical physiological parameter measurements, and/or may further perform digital signal filtering, signal quality determination and classification, or any combination of thereof. Such pre-processing may generate metrics that include historical and/or real-time parameter trends, detected parameter patterns, parameter variability calculations, and signal quality indicators, to name a few non-limiting examples. Trend metrics may indicate if a physiological parameter is increasing or decreasing at a certain rate of change over a certain duration, pattern metrics may indicate if a parameter is periodic over a particular duration or within a specified frequency range, and variability metrics may indicate the extent of physiological parameter stability.

The sepsis monitor 106 may include a metric analyzer configured to provide test results and supplemental metrics to post-processing components based upon various rules applied to the physiological parameter metrics in terms of various thresholds, patterns, and expected ranges. As an example, the metric analyzer may output an alarm trigger when a parameter measurement increases faster than a predetermined expected rate. This may be expressed, as an example, by a rule that states, “if the metric trend exceeds the trend threshold, trigger the alarm.” Tables 1 and 2 below relate to sepsis monitor rules applied to metrics including Heart Rate, Respiratory Rate, Temperature, and Blood Pressure parameters and trends. However, other patient parameters may be used in detecting and quantifying sepsis.

The post-processing component of a multiparameter sepsis monitoring algorithm may process various other test results and metrics that the sepsis monitor 106 does not directly monitor (e.g., blood test results). Such other test results and metrics may augment the sepsis classification, score, index and/or other metrics to grant further reliability and confidence in the diagnosis and quantification of the sepsis state of the patient 104. The outputs from post-processing by the sepsis monitor 106 may include alarms, controls, and/or diagnostics. As an example, alarms may include audible and/or visual cues, such as an intermittent tone or a green/yellow/orange/red color or similar icons, indicating a patient's sepsis state or other diagnostic information. Diagnostics may indicate a particular patient condition, such as the potential onset of sepsis or other physiological conditions that the device is able to monitor. Controls may be electrical or electronic, wired and/or wireless, or mechanical, and may be capable of interfacing with and affecting other devices. Embodiments of a sepsis monitor 106 are described in more detail below.

The processing circuitry 108 may include hardware and/or software for carrying out computing tasks, for example, tasks associated with monitoring sepsis in a patient 104, providing corresponding audio and/or visual alerts, and/or providing control signals for the purpose of treating the patient 104. For example, the processing circuitry 108 may comprise hardware, such as an application specific integrated circuit (ASIC). Other non-limiting examples of the processing circuitry include an Integrated Circuit (IC) chip, a Central Processing Unit (CPU), a Graphics Processing Unit (GPU), a microprocessor, a Field Programmable Gate Array (FPGA), a collection of logic gates or transistors, resistors, capacitors, inductors, diodes, or the like. Some or all of the processing circuitry 108 may be provided on a Printed Circuit Board (PCB) or collection of PCBs. It should be appreciated that any appropriate type of electrical component or collection of electrical components may be suitable for inclusion in the processing circuitry 108. In one example, the processing circuitry includes one or more microprocessors that execute instructions stored on memory 124 and/or stored on other memory local to the processing circuitry 108. In general, the processing circuitry 108 controls the interactions between the elements of the system 100 and may perform one or more of the methods described herein.

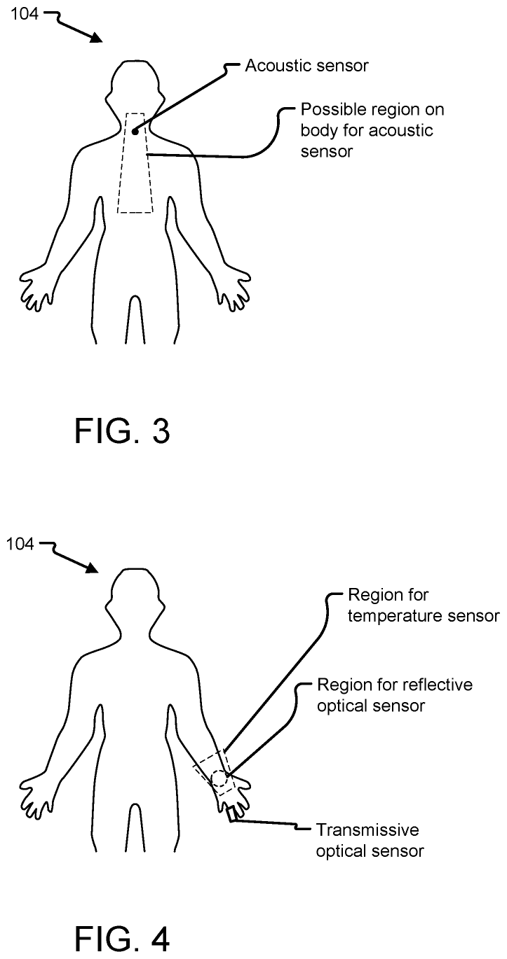

The one or more sensors 112 may include one or more noninvasive sensors that monitor various physiological parameters of the patient 104. Ex vivo sensors may be considered noninvasive while in vivo sensors may be considered invasive. For example, the sensors 112 may include sensors suitable for noninvasively monitoring patient temperature, blood analytes, respiration rate, blood pressure, heart rate, brain activity, blood oxygen saturation, perfusion index, and/or any other physiological parameter of the patient 104 that may be relevant to making a sepsis determination and/or relevant to the patient's 104 health. Such sensors 112 may include optical sensors (e.g., optical emitters and receivers of a pulse oximeter), electrical sensors (e.g., electrodes that induce and/or sense electrical signals), mechanical sensors, electromechanical sensors (e.g., blood pressure cuff), and/or the like. FIGS. 3-6 illustrate specific, non-limiting, examples of sensors 112.

The output device(s) 116 may include any suitable hardware and/or software for producing an audio alarm and/or a visual alarm to provide state information on one or more physiological parameters of the patient 104. For example, an output device 116 may include one or more displays for displaying the state information. An output device 116 may include one or more speakers that output various audible alarms based on the state information. The visuals and/or audio output by the output device 116 may change as the physiological parameters of the patient 104 change.

The input device(s) 120 may include any suitable hardware and/or software for accepting and/or processing user input. For example, an input device 120 may include a mouse, a keyboard, a touch screen or touch sensitive portion of a display, a microphone, mechanical buttons, electromechanical buttons, and/or the like.

In at least one embodiment, the input device(s) 120 and the output device(s) 116 may be incorporated into one or more user interfaces that enable a combination of user inputs and outputs described above.

The memory 124 may correspond to any suitable type of non-transitory computer-readable medium. In some embodiments, memory 124 may comprise volatile or non-volatile memory and a controller for the same. Non-limiting examples of memory 124 include RAM, ROM, buffer memory, flash memory, solid-state memory, and/or variants thereof. In at least one embodiment, the memory 124 includes instructions that are executed by the processing circuitry 108 to monitor for possible sepsis in the patient 104 in accordance with one or more of the methods described herein. The memory 124 may be local to or remote from the processing circuitry 108. The memory 124 may store data associated with monitoring sepsis (e.g., sensor data from sensors 112, historical patient data, and/or the like).

The database(s) 128 may be any suitable type of non-transitory computer-readable medium and may have the same or similar structure as the memory 124 described above. In at least one embodiment, a database 128 is remote from the other elements of the system 100 and may store data suitable for use before, during, and/or after monitoring the patient 104 for sepsis. In one non-limiting example, a database 128 serves as central storage for multiple systems 100. For instance, a database 128 may include sepsis monitoring data for multiple patients 104, where such sepsis monitoring data may be fed into a neural network 136 as training data from the database 128 (and/or from the processing circuitry 108) that executes one or more machine learning algorithms to improve the accuracy of sepsis detection and corresponding sepsis treatments for future patients 104. Such machine learning algorithms may include linear regression algorithms, logistic regression algorithms, decision tree algorithms, k-means algorithms, and/or the like. The neural network 136 may include processing circuitry the same as or similar to the processing circuitry 108 and implement one or more methods of learning, such as supervised learning, unsupervised learning, reinforcement learning, self-learning, and/or the like.

The additional devices 132 may comprise any suitable device or set of devices for monitoring other states of the patient 104 besides sepsis and/or any suitable device or set of devices for providing treatment to the patient 104. Additional devices for monitoring may include devices and/or sensors that are considered useful for maintaining or improving the health of the patient 104 but not necessarily useful for monitoring sepsis as are the sensors 112. Such additional devices and/or sensors 132 for monitoring the patient 104 may be invasive and/or noninvasive in nature. Devices for providing treatment to the patient 104 include devices that monitor fluid in the patient 104 and/or devices that provide fluids (e.g., antibiotics, saline, and/or the like) to the patient 104 in accordance with the patient's 104 state (e.g., sepsis state).

Although not explicitly shown, each element in the system 100 may include one or more communication interfaces for communicating with one another over suitable wired and/or wireless connections. In addition, it should be appreciated that the system 100 may include other processing devices, storage devices, and/or communication interfaces generally associated with computing tasks, such as sending, receiving, and processing data related to the care of the patient 104.

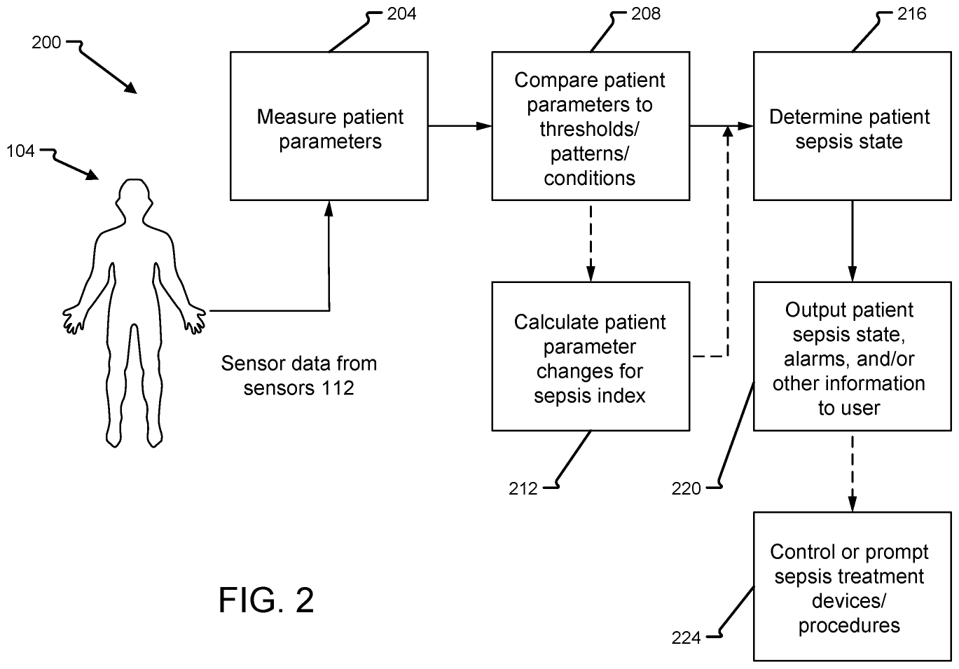

FIG. 2 illustrates a general method 200 for the sepsis monitoring system 100 having one or more physiological sensors 112 generating sensor signals for monitoring sepsis, where the generated sensor signals are used to determine the state of sepsis onset in a patient 104.

With reference to FIG. 2, operation 204 may include measuring patient parameters based on sensor data from sensors 112. For example, the sensors 112 may output signals that are indicative of one or more physiological parameters of the patient 104, such as respiration rate, heart rate, temperature, blood pressure, blood analytes, and/or the like, which are translated into actual measurements by the processing circuitry 108 according to suitable methods. In one embodiment, sensor data corresponds to data derived from raw sensor signals output from the sensors 112 themselves. For example, the sensor data may correspond to raw sensor signals that have undergone one or more modifications (e.g., signal amplification, sampling, etc.) at the sepsis monitor 106. In at least one embodiment, sensor data is simply the raw sensor signals as output from the sensors 112 (i.e., the sensor data and the sensor signals are the same).

Operation 208 includes comparing the patient parameters from operation 204 to one or more thresholds, one or more patterns, and/or one or more conditions, where such thresholds, patterns, and/or conditions are used to determine a sepsis state of the patient 104 in operation 216. Operation 212 may be optionally performed to calculate patient parameter changes for the purpose of determining a sepsis index, where such sepsis index is used to determine the sepsis state of the patient 104.

Operation 220 includes outputting the sepsis state of the patient 104. For example, the output device 116 may output one or more audio and/or visual alarms along with other information to a user of the sepsis monitor 106. The other information may include information about sepsis trends (e.g., sepsis state improving or worsening), suggested treatment options, and/or the like.

Operation 224 is optional and includes controlling or prompting sepsis treatment devices and/or sepsis treatment procedures based on the patient's 104 sepsis state. As may be appreciated, the operations depicted in FIG. 2 are described in more below with reference to FIG. 3 onward.

FIGS. 3-6 illustrate various examples for noninvasive sensors 112 used within the sepsis monitor 206 as well as and potential locations on the patient 104 for each sensor.

In more detail, FIG. 3 illustrates an acoustic sensor, which may function as a respiration sensor for detecting a respiration rate of the patient 104. One possible region for placing the acoustic sensor on the patient 104 extends from the patient's neck area to the patient's mid-torso. The acoustic sensor may sense body-sounds during patient breathing and output signals to measure respiration rate (RR). In one embodiment, the respiration sensor comprises an optical sensor to generate the same respiration rate parameter in response to the optical sensor signals. An acoustic sensor is described in U.S. Pat. No. 6,661,161 entitled Piezoelectric Biological Sound Monitor with Printed Circuit Board and a corresponding respirator rate monitor is described in International App. No. PCT/CA2005/000536 and Pub. No. WO 2005/096931, filed Apr. 8, 2005, both applications incorporated by reference herein.

FIG. 4 illustrates a possible region on the patient 104 for placing a sensor that measures temperature of the patient 104, where such region is between the wrist and elbow of the patient 104. A temperature parameter is generated via the temperature sensor, which may be optical or electromechanical. Alternatively, patient temperature is manually entered or provided to the sepsis monitor 106 from another source.

FIG. 4 further illustrates a possible region for a reflective optical sensor located at a wrist of the patient and a possible region for a transmissive optical sensor on a digit of the patient 104. As may be appreciated, these optical sensors may generate signals for measuring pulsatile-blood related parameters, such as Heart Rate (HR) and blood pressure, along with blood analyte measurements (e.g., Methemoglobin, Carboxyhemoglobin, Total Hemoglobin, Hematocrit, Deoxygenated Hemoglobin, Oxygenated Hemoglobin, Bilirubin, Oxygen Content (O₂ct) and Saturation Percent of Oxygen (SpO₂), perfusion index, and/or the like) based on amounts of light absorbed by patient tissue.

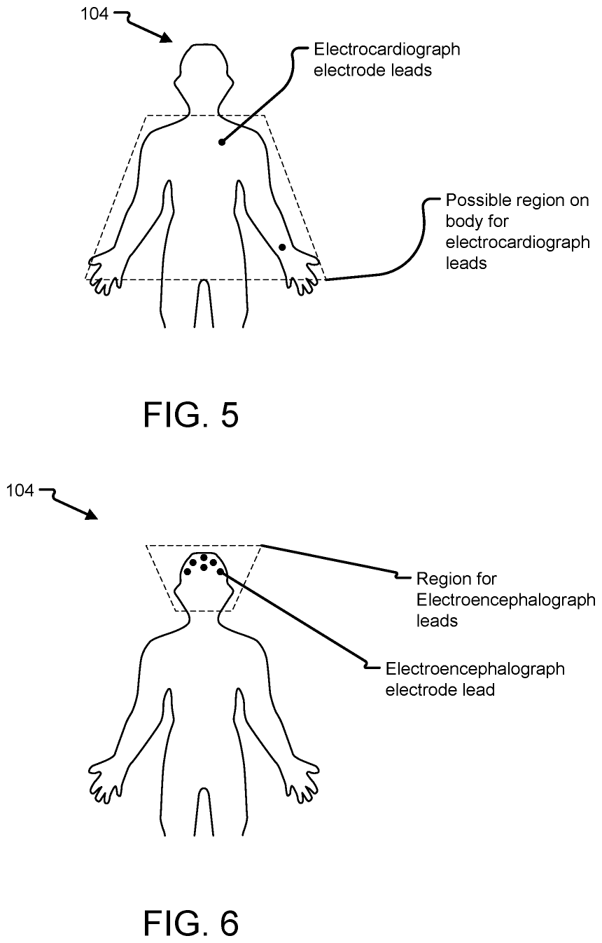

FIGS. 5 and 6 illustrate electrodes attached to a patient 104 for measuring various physiological parameters. The electrodes in FIG. 5 may comprise electrodes for electrocardiograph (ECG) measurements and may be positioned at one or more locations on the thoracic and/or arms of the patient. Meanwhile, the electrodes in FIG. 6 serve as electrodes for encephalograph (EEG) measurements and may be placed in a region that corresponds to the scalp or cranium of the patient 104. A multiple parameter processor, such as processing circuitry 108, analyzes the parameter measurements, alone or in combination, and generates sepsis indicators and alarms or sepsis treatment controls, or both, in response to the measured parameters.

In at least one embodiment, a multiple parameter processor, such as the processing circuitry 108, is responsive to a combination of multiple physiological parameters to indicate sepsis, making it advantageously more sensitive and specific to presentations of sepsis conditions to a degree further than any similar single-parameter noninvasive monitor. Further, the multiple parameter processor responds not only to parameter limits but also to parameter trend information, parameter patterns, and parameter variability, so as to reflect patient sepsis conditions over time. In an embodiment, sepsis indicators include alarms and indications that designate stages of sepsis: none, onset of sepsis, severe sepsis, or septic shock. These outputs, for example, provide a warning of a potential onset of sepsis at an early stage and can trigger alarms as sepsis symptoms progress. In other embodiments, the multiple parameters could be synthesized into a singular representation of the analyzed parameters (limits, rising and falling trends, empirical measurements) being presented as; a combined index value where the index represents changes in the combination of measured parameters that are indicative of the onset of sepsis, or a set of quantized stages ranging from no sepsis to septic shock, or other. The combined sepsis state or index value may be the basis for generating indicators and alarms. Further, administration of drug or other therapies or alteration of drug doses can be controlled in response to the patient condition being quantified by the index value. Additionally, or alternatively, sepsis may be determined according to a threshold-based metric analysis, as described in more detail below with respect to Tables 1 and 2 and corresponding figures.

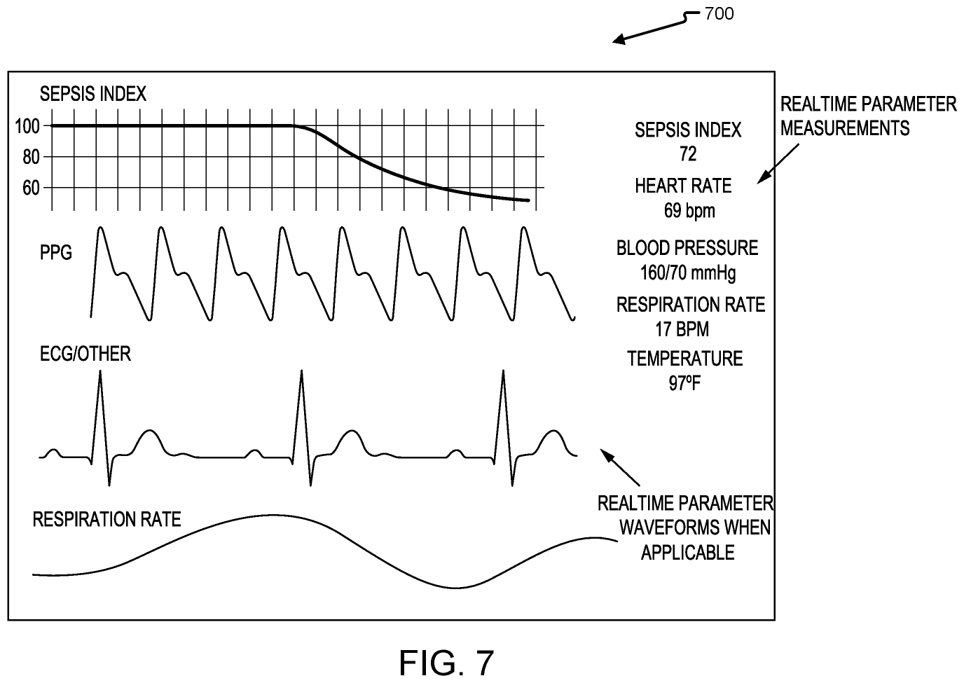

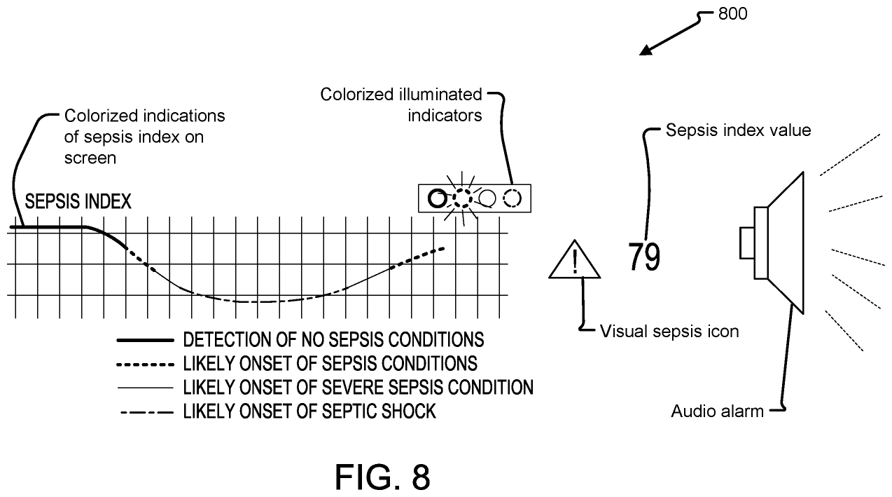

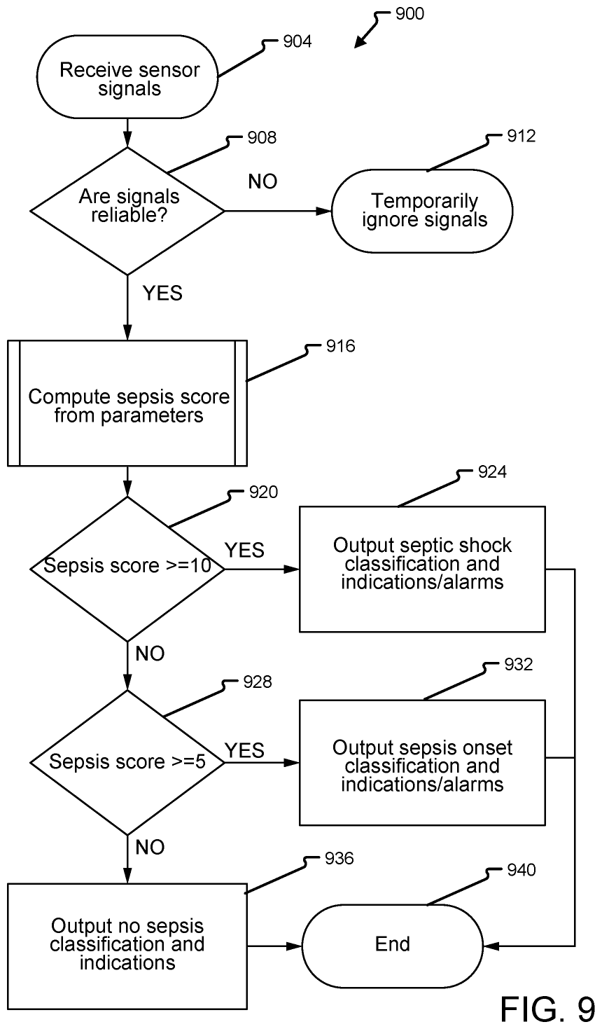

FIGS. 7 and 8 illustrate example graphics 700 and 800 on a display of a sepsis monitor 106 according to at least one example embodiment. The display of the sepsis monitor 106 may be included in the output device(s) 116.

As shown in FIG. 7, the sepsis monitor 106 may display a sepsis index of a patient 104 over time, one or more real time parameter waveforms of the patient 104 (e.g., PPG waveform, respiration rate waveform, and an ECG or other waveform), and one or more real time parameter measurements of the patient 104 (e.g., a current sepsis index value of the patient 104, patient heart rate, patient blood pressure, patient respiration rate, and patient temperature). The graphic 700 may include diagnostic messages or other indicators used to assist medical personnel in diagnosing and treating a patient's condition(s). Although not explicitly shown, other outputs of the sepsis monitor 106 may include control signals that affect the operation of a medical treatment device or subsystem for treating sepsis in the patient 104. FIG. 8 illustrates additional details of the sepsis index graphic from FIG. 7.

Whether using the sepsis index value or a threshold-based sepsis analysis (see TABLES 1 and 2 below) to determine the sepsis state of a patient 104, a sepsis monitor 106 may include various external indicators and/or alerts as shown in FIG. 8. In one embodiment, the colorized sepsis indicators may be included with the index tracker itself and/or separate from the index tracker. In any event, a green indicator may signal a stable condition with no sepsis conditions being detected (thick solid line), a yellow indicator may signal potential onset of sepsis conditions (dashed line), an orange indicator may signal the onset of more severe sepsis conditions (thin solid line), and a red indicator may signal a severe sepsis condition, such as septic shock (dashed line of different lengths). The indicators may be, for example, various display LEDs emitting wavelengths of the appropriate colors, or an on-screen display featuring coloration, text, or icons as indications of sepsis state of the patient. As indicate in FIG. 8, audible alarms may be emitted with tones corresponding to the state of sepsis onset.

There are additional physiological indicators of sepsis which are not measured non-invasively, such as various markers and indications determined through blood testing. Thus, in at least one embodiment, the sepsis monitor 106 prompts healthcare providers to obtain various additional indicators of sepsis, such as blood tests, which the device would not itself monitor non-invasively, with the test results or physiological parameters being input into the sepsis monitor 106 either manually by a healthcare provider or via a patient electronic medical records system (e.g., from database 128) which the sepsis monitor 106 is in communication with, automatically or manually.

Example embodiments are discussed in more detail below with reference to selected parameters for non-invasive detection of sepsis in a patient that include heart rate, respiration rate, temperature, and blood pressure. However it should be appreciated however that the techniques described herein may be practiced with a variety of suitable physiological parameters, vital signs, blood analytes, and/or other indicators for detecting septic state and the onset of sepsis in a human.

Table 1 below relates to a threshold-based classification of sepsis in a patient 104. For example, with reference to Table 1, if a patient's heart rate (HR) and respiration rate (RR) are less than predetermined maximum limits and their body temperature is within a predetermined normal range, then the sepsis monitor 106 displays a no sepsis condition (green or similar) indicator. However, if more than one of heart rate, respiration rate and body temperature, or other parameters are changing, where applicable changes in heart rate and respiration rate are increased beyond normal ranges, then the sepsis monitor 106 displays a potential onset of sepsis (yellow or similar) indicator. If more than one of heart rate, respiration rate and temperature, or other, parameters become abnormal, including heart rate and respiration rate above a predetermined limit and temperature outside of a predetermined range, then the sepsis monitor 106 displays potentially more severe sepsis conditions (orange or similar) indicator. Similarly, if more than one of heart rate, respiration rate and temperature, or other, parameters become critical or severely abnormal, including heart rate and respiration rate above a predetermined limit and temperature outside of a predetermined range, then the sepsis monitor 106 signals the onset of septic shock conditions (red or similar).

TABLE 2

| | |
|---|---|
| | SEPSIS SCORE |
| RULE | CONTRIBUTION |

Heart Rate (bpm)

| | |
|---|---|
| HR < +25% of baseline HR | +0 |
| HR < +40% of baseline HR | +1 |
| HR ≥ +40% of baseline HR | +2 |

Respiration Rate (bpm)

| | |
|---|---|
| RR < 22 | +0 |
| RR ≥ 22 | +2 |

Temperature (° F.)

| | |
|---|---|
| 97.3 ≥ Temp ≤ 99.0 | +0 |
| Temp < 97.3 OR Temp > 99.0 | +1 |
| Temp < 96.8 OR Temp > 100.4 | +2 |

Systolic Blood Pressure (mmHg)

| | |
|---|---|
| SBP > 100 | +0 |
| SBP < 100 | +2 |
| SBP < 80 | +4 |

Presence of Sepsis Indicators in Blood
Test Results (measured independently)

| | |
|---|---|
| No | +0 |
| Yes | +4 |

In Table 1 above, a nominal parameter limit may be a value or range of values for a parameter that is considered normal for a particular patient. Similarly, an abnormal parameter limit may consist of a value or range of values for a parameter that is outside the nominal value or range of values for a particular patient but is not yet in a critical limit. A critical parameter limit may be a value or a range of values for a parameter that is outside the values or ranges that are nominal and abnormal. On a scale of medical severity, exceeding a critical limit is more severe than exceeding an abnormal limit, and exceeding an abnormal limit is more severe than exceeding a nominal limit. The values or ranges of values for nominal, abnormal, and critical limits may vary according to characteristics of the patient (e.g., patient's age, medical history, height, weight, gender, etc.) or may be derived and adjusted from the patient's measured baseline (normal and non-septic) values, or a combination of both.

Table 1 demonstrates a sample set of conditions that may be used for classification of sepsis, where the conditions are bound to a set of thresholds, either static or dynamically set or adjusted based on the patient 104 and/or on measured physiological parameters of the patient 104. As may be appreciated, two mechanisms for the conditional classification may be applied to the measured physiological parameters: implementing the condition definitions as in Table 1 (e.g., IF heart rate is greater than 80 bpm AND EITHER respiration rate is greater than 30 bpm OR temperature is greater than 100 degrees Fahrenheit . . . Classification: likely onset of sepsis condition); or implementing a composite value as in Table 2, where for each individual parameter and for every additional level of sepsis indication, confidence values are used to generate a sepsis score that is indicative of the sepsis state of the patient 104.

With reference to Table 2 below, baseline values for physiological parameters may be set for a particular patient 104 and used to determine an overall sepsis score. For example, when heart rate is greater than 80 bpm, +1 is added to an overall sepsis score. As another example, +2 is added to the sepsis score for a heart rate that is greater than 90 bpm. In yet another example, a heart rate of less than 70 bpm may have no impact while Respiration Rates over 25 bpm may add +2 to the sepsis score and so on for all the measured physiological parameters. Each confidence value may be based on the significance and confidence of the presenting physiological parameters and conditions being monitored. As shown in Table 3, the overall sepsis score may then be broken down into categories based on ranges of the score: for example, no likely sepsis conditions for a score of 0-4, possible onset of sepsis for a score of 5-7, severe sepsis for a score of 8-9, and septic shock for a score of 10 or more. Additionally, at least one example embodiment may implement both explicit conditional rules in Table 1 and the composite indication scoring in Table 2.

TABLE 3

| | SEPSIS STATE |
|---|---|
| SEPSIS SCORE | CLASSIFICATION |
| 0-4 | Sepsis Not Likely |
| 5-7 | Onset of Sepsis |
| 8-9 | Severe Sepsis |
| 10 or more | Septic Shock |

TABLE 4

| | SEPSIS STATE |
|---|---|
| SEPSIS INDEX | CLASSIFICATION |
| 100-90 | Sepsis Not Likely |
| 90-80 | Onset of Sepsis |
| 80-70 | Severe Sepsis |
| 70 or less | Septic Shock |

FIG. 9 illustrates a method 900 according to at least one example embodiment. The method 900 may be used to compute a sepsis score according to Table 2.

In operation 904, a sepsis monitor 106 may receive sensor signals from sensors 112 that are indicative of a patient's 104 physiological parameters. The physiological parameters being monitored may be the same as those discussed above with reference to Tables 1 and 2, but may also include additional parameters not in Tables 1 and 2 (e.g., blood analytes, blood oxygen saturation, ECG measurements, EEG measurements, etc.).

Operation 908, includes determining whether the sensor signals or sensor data derived from the sensor signals are reliable. If not, values of the physiological parameters derived from the sensor signals may be temporarily ignored in operation 912. Sensor signals or sensor data may be unreliable in cases such as if the patient 104 moves, which makes measurements such as the temperature or heart rate temporarily unreliable. Reliability of a portion of a signal may be determined by examining portions of the signal immediately preceding and immediately following the portion of the signal in question (e.g., within a few seconds on either side of the portion of the signal in question). The portion of the signal in question may be determined to be unreliable if one or more signal characteristics (e.g., SNR, amplitude, frequency, phase) of the portion in question do not sufficiently match the portions of the signal immediately preceding and following the portion of the signal in question. In this case, a mismatch may indicate a temporary inaccurate reading from a monitor due to patient or monitor movement or other cause. Stated another way, if the signal characteristics of the portion of the signal in question exceed preset thresholds or ranges, then the portion of the signal in question may be determined as unreliable. In at least one embodiment, unreliable signals may be determined from the measured physiological parameters. For example, if the patient's measured heart rate experiences a sudden spike or drop that exceeds a preset threshold, the portion of the sensor signal that caused the sudden spike or drop may be determined as unreliable. In at least one embodiment, unreliable sensor signals or measured physiological parameters may be corrected or adjusted, if possible. For example, an unreliable portion of a sensor signal may be averaged with surrounding reliable portions of the sensor signal or the unreliable portion may be replaced by a reliable portion. In another example, a measured physiological parameter that is determined to be unreliable for a time period may be averaged with the measurements before and/or after the unreliable measurement or the unreliable measurement may be replaced with a reliable measurement that is temporally adjacent to the unreliable measurement.

Returning to operation 908, if the sensor signals or sensor data are determined to be reliable, the reliable sensor signals or sensor data are used to compute a sepsis score from physiological parameters derived from the sensor signals in operation 916. The sepsis score may be computed in the same or similar manner as that discussed above with reference to Table 2 where the measured physiological parameters are compared to baseline values that are specific to the patient 104.

Operation 920 includes determining whether the computed sepsis score is greater than or equal to a first threshold, in this case, the value 10. If so, the method 900 proceeds to operation 924 to output an indication of septic shock and corresponding alarms and/or control signals for implementing treatment. If not, the method 900 proceeds to operation 928 and determines whether the sepsis score is greater than or equal to a second threshold, for example, the value 5. If the sepsis score is greater than or equal to the second threshold value, the method 900 proceeds to operation 932 to output an indication that the patient 104 may be experiencing the onset of sepsis. If not, the method 900 proceeds to operation 936 and outputs an indication that sepsis likely does not exist in the patient 104. The method 900 ends at operation 940.

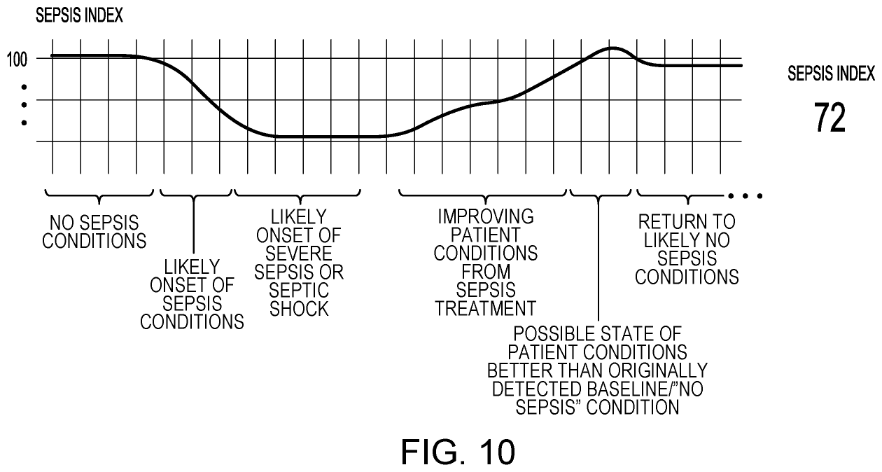

In at least one example embodiment, a sepsis monitor 106 may produce a continuous “sepsis index” value, which would be displayed to the user. Such a sepsis index value may be based on changes in each monitored parameter as compared to a corresponding baseline value. The baseline values may be recorded or set at the start of patient monitoring while no sepsis conditions are present. As the physiological parameters of the patient change, the magnitude of the change is compounded with a designated multiplier unique to the measured parameter. The designated multiplier may be based on the measured parameter's reliability for indicating the onset of sepsis conditions. This multiplied value, along with the similar values for all other measured parameters, are then added/subtracted from the nominal ‘Sepsis Index’ starting value, such as 0 or 100. This method produces an index which starts at a designated nominal value for all patients that corresponds to their baselines and tracks along with changes in the aggregate of parameters being monitored to indicate sepsis, as illustrated in FIG. 10. In at least one embodiment, the calculated Sepsis Index value may be used for classification of the onsetting of sepsis, as shown in Table 4. Parameter coefficients, starting baseline index value, and other components used in the algorithm for calculating a sepsis index are not limited to these example values, limits, constraints, magnitude, or scope.

TABLE 1

| RULE | OUTPUT |
|---|---|
| Patient Heart Rate is within Nominal Heart | Indicate nominal/no |
| Rate Limit | sepsis condition |
| Patient Respiration Rate is within Nominal | (green or similar) |
| Respiration Rate Limit, while Temperature is | |
| within nominal range | |
| Patient Heart Rate > Nominal Heart Rate | Indicate potential onset |
| Limit | of sepsis conditions |
| Patient Respiration rate > Nominal | (yellow or similar) |
| Respiration Rate Limit, while Temperature is | |
| above OR below nominal temperature limits. | |
| Current Heart Rate > abnormal Heart Rate | Indicate severe sepsis |
| limit | (orange or similar) |
| Respiration Rate > abnormal Respiration Rate | |
| limit, while Temperature is above OR below | |
| abnormal Temperature limits. | |
| Current Heart Rate > critical Heart Rate limit | Indicate septic shock |
| Respiration Rate > critical Respiration Rate | (red or similar) |
| limit, while Temperature is above OR below | |
| critical Temperature limits. | |

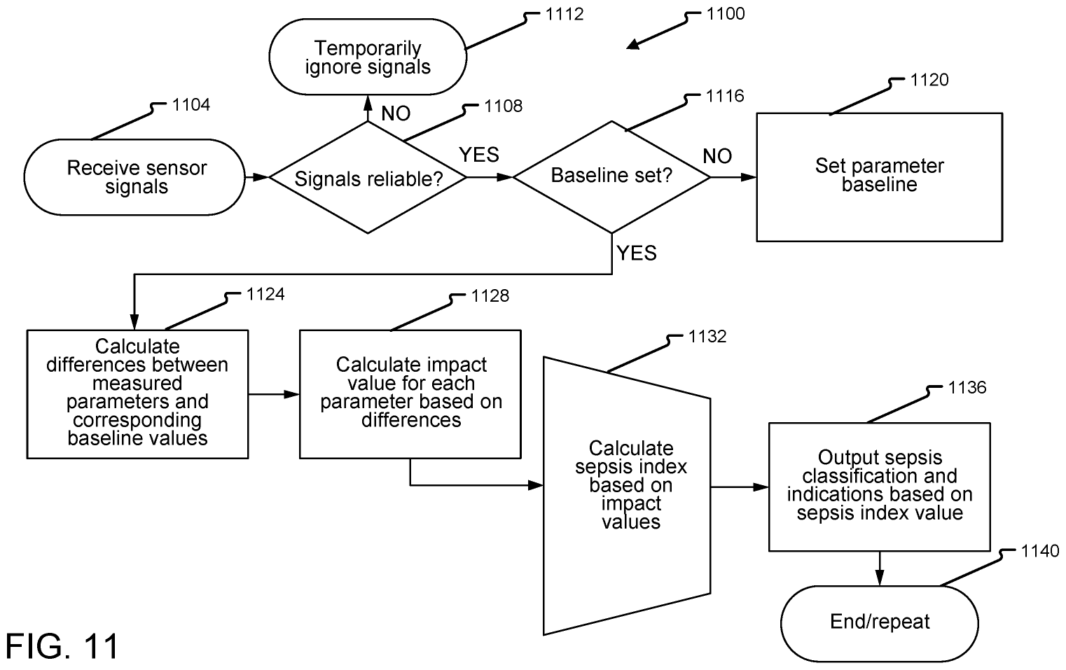

FIG. 11 illustrates a method 1100 for generating a sepsis index according to at least one example embodiment. In more detail, FIG. 11 relates to an algorithm for determining the sepsis index as derived from the combination of multiple physiological parameters which are, individually, indicators of sepsis, so as to produce a singular scoring mechanism for sepsis conditions in a patient. The result is a sepsis monitor 106 that features a high degree of confidence and specificity as a result of using a combination of multiple parameters as indicators for sepsis conditions.

Operation 1104 includes a sepsis monitor 106 receiving or generating sensor data, where such sensor data may be based on raw sensor signals output by sensors 112 that are noninvasively monitoring a patient 104. Operation 1108 includes determining whether the sensor data and/or the sensor signals are reliable, for example, in the same or similar manner as that described above with respect to FIG. 9. If not, the method proceeds to operation 1112 to temporarily ignore the unreliable sensor signals. Alternatively, the unreliable sensor signals may be corrected or adjusted if possible. If the sensor signals are reliable in operation 1108, the method 1100 proceeds to operation 1116 to determine whether baseline values exist for the physiological parameters being monitored. If not, operation 1120 sets the baseline values for the patient 104. In general, the baseline value for each monitored parameter corresponds to a normal value for the patient 104 when no sepsis is present. The baseline values may be specific to the patient 104 based on currently measured physiological parameters, patient age, gender, pre-existing medical conditions, and/or the like.

If operation 1116 determines that the baseline values for the monitored parameters are set, then the method 1100 proceeds to operation 1124 to calculate differences or changes between each monitored parameter and corresponding baseline value.

Operation 1128 includes calculating an index impact value from the difference between each parameter and its corresponding baseline value.

Operation 1132 includes calculating the sepsis index based on impact values determined in operation 1128. The calculations in operations 1128 and 1132 are discussed in more detail below with respect to a specific, non-limiting, example.

Operation 1136 includes determining and outputting a sepsis classification or sepsis state of the patient 104 along with one or more alarms and/or control signals based on the sepsis index determined in operation 1132. The method 1100 ends or repeats at operation 1140.

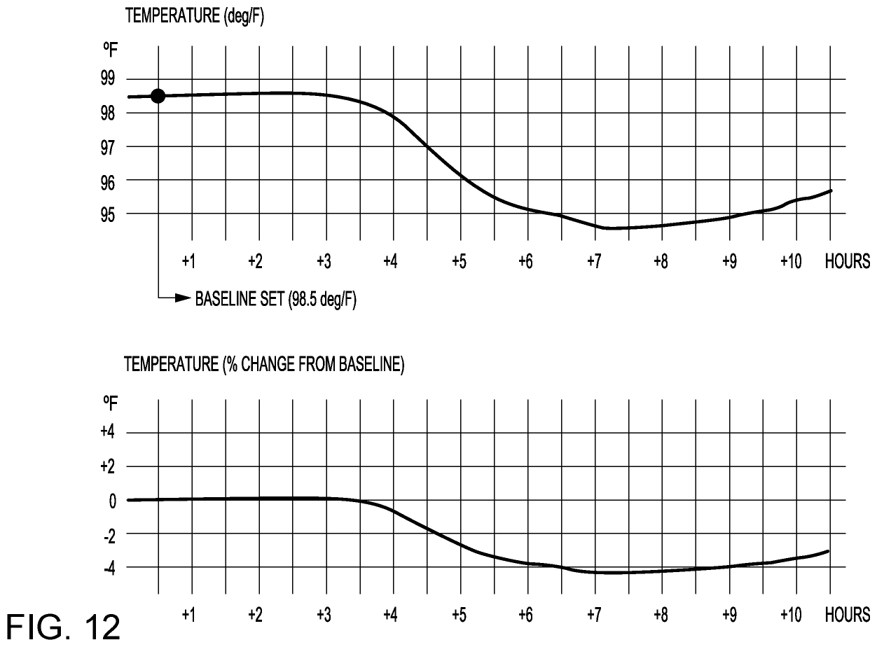

The method 1100 will now be described with reference to a specific example in which the sepsis index begins at a value of 100 (i.e., baseline values are set for a particular patient 104 such a sepsis index value of 100 indicates a state of no sepsis or onset of sepsis for the patient 104). Each monitored physiological parameter has an associated index coefficient (or multiplier) which is used to provide an appropriate weight of significance for the overall sepsis index value. As each monitored parameter changes away from its baseline, a percentage change is calculated (operation 1124) and the percentage change is multiplied by the parameter's associated coefficient to determine the parameter's impact on the sepsis index (operation 1128). FIGS. 12-15 illustrate an example of the sepsis index computation happening at +5 hours into a monitoring session.

As shown in FIG. 12, for example, temperature decreases from the baseline of 96.5° F. by −2.5%, which is multiplied by a coefficient of 240 to result in an impact value of −6 on the overall index:

{240*−0.025=−6}, at t=+5 hours

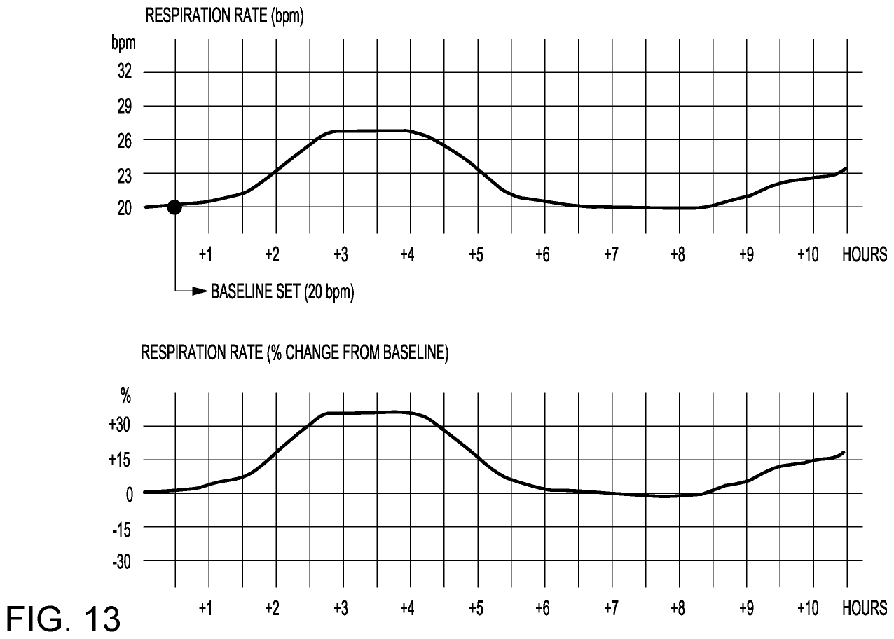

Additionally, as shown in FIG. 13, respiration rate increases from the baseline of 20 bpm by +15%, which is multiplied by a coefficient of −80 to result in an impact value of −15 on the sepsis index:

{−80*0.15=−12}, at t=+5 hours

Combined, both temperature and respiration rate have a combined Sepsis Index Impact of −18 to be added to the starting index value of 100 to produce the calculated Sepsis Index of 82 at 5 hours elapsed (in this example):

{100+(−6)+(−12)=82}, at t=+5 hours

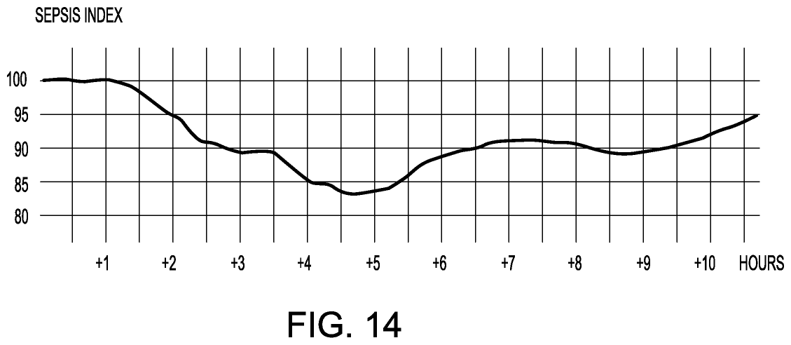

Still with reference to this specific example, FIG. 14 illustrates the sepsis index changing over time where the sepsis index is 82 at 5 hours into a monitoring session. While the above example only uses two parameters for simplicity of explanation, all parameters being measured may have an associated sepsis index impact coefficient that influences the overall sepsis index. The coefficients mentioned above may be determined based on the parameter's reliability for indicating the onset of sepsis conditions. The coefficients may be fixed for the entire monitoring session or vary (according to one or more predetermined mathematical functions) over a monitoring session and/or between different monitoring sessions. For example, the coefficient for a particular parameter may change if the parameter's reliability for indicating the onset of sepsis changes. Additionally, coefficients may change based on interdependencies based on measurements and patterns of multiple measured parameters. The coefficients and/or functions that determine the coefficients may be determined based on historical data, real-time data, or both. Examples of historical data include data collected from a previous sepsis monitoring session or other medical monitoring of a patient and/or data from monitoring sessions of other patients. Examples of real-time data include data collected during the current monitoring session of a patient and/or data collected during concurrent monitoring sessions of other patients. The historical data and real-time data may be processed by an machine learning system or neural network (e.g., neural network 136) that is programmed to improve the coefficients or the functions of the coefficients using the historical data and/or real-time data. The neural network may employ one or more machine learning algorithms, which may include supervised learning, unsupervised learning, reinforcement learning, deep learning, and/or the like of similar techniques.

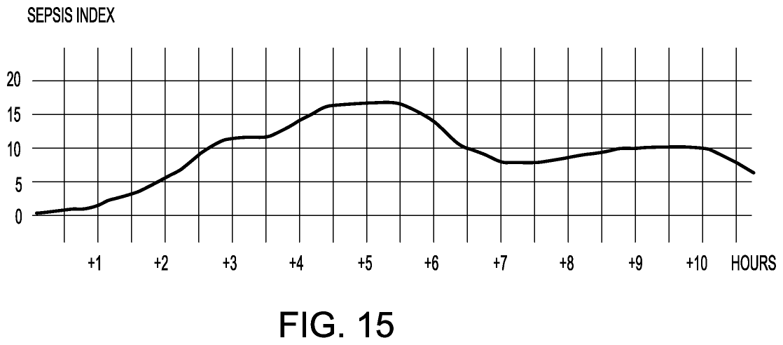

FIG. 15 illustrates an example where the initial sepsis index value is 0 instead of 100 (i.e., the baseline with no presenting sepsis conditions from the measured parameters or their relative changes produces a Sepsis Index value of 0). As sepsis conditions compound in a patient, the index will increase above 0 to indicate the onsetting sepsis conditions and will return towards 0 as sepsis indications dissipate, as signaled by the parameters' return towards their baseline values. In at least one embodiment, the sepsis index may have values below 0 (or above 100 in FIG. 14), which may signify that the patient has measured parameters that are better than the original baseline values (in the context of sepsis conditions and diagnosis). As described in other embodiments, the sepsis index can be stylized (i.e., colored) in relation to Table 4 as a means of sepsis state indication.

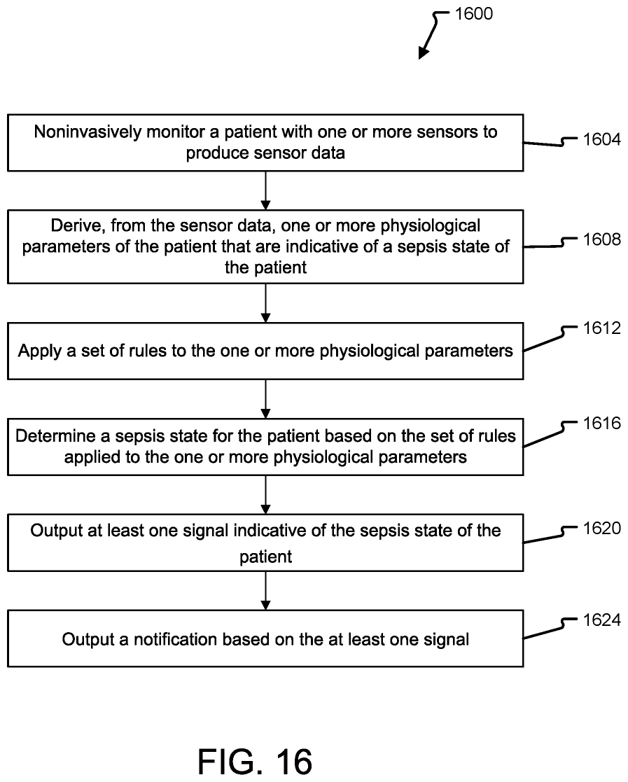

FIG. 16 illustrates a method 1600 according to at least one example embodiment. The method 1600 relates to detecting sepsis or the onset of sepsis in a patient 104 using a sepsis monitor 106 as discussed with reference to FIGS. 1-15 above.

Operation 1600 includes noninvasively monitoring a patient 104 with one or more sensors 112 to produce sensor data or sensor signals. In one non-limiting example, the one or more sensors 112 include a heart rate sensor, a temperature sensor, and a respiration rate sensor. However, example embodiments are not limited thereto, and other sensors may be included depending on the type of physiological parameters being monitored.

Operation 1608 includes deriving, from the sensor data, one or more physiological parameters of the patient that are indicative of a sepsis state of the patient 104. For example, the processing circuitry 108 receives the sensor data and translates the sensor data into values for the one or more physiological parameters, which may include heart rate monitored by the heart rate sensor, temperature monitored by the temperature sensor, and respiration rate monitored by the respiration rate sensor. However, example embodiments are not limited thereto, and other physiological parameters relevant to a sepsis determination may be derived from the sensor data.

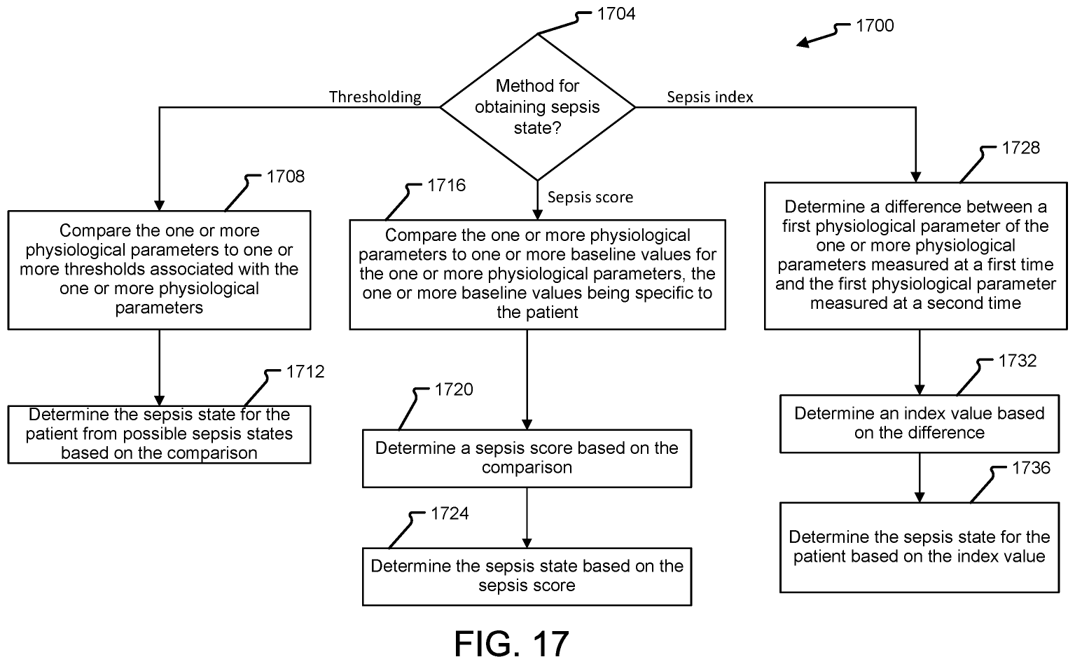

Operation 1612 includes applying a set of rules to the one or more physiological parameters while operation 1616 includes determining a sepsis state of the patient 104 based on the set of rules applied to the one or more physiological parameters. Operations 1612 and 1616 are discussed in more detail below with reference to FIG. 17.

Operation 1620 includes outputting at least one signal indicative of the sepsis state of the patient while operation 1624 includes outputting a notification based on the at least one signal. For example, the notification may comprise one or more audio/visual alarms on an output device 116 to alert a medical professional of the patient's 104 sepsis state. Operation 1620 may further include outputting one or more control signals to a treatment device 132 to cause the treatment device 132 to begin treating the patient 104 for sepsis and/or outputting one or more suggestions for how to treat the patient 104.

FIG. 17 illustrates a method 1700 that provides further details for operations 1612 and 1616 from FIG. 16.

Operation 1704 includes determining the method for obtaining the patient's 104 sepsis state. As discussed above with reference to Tables 1 to 4 and related figures and text, the patient's sepsis state may be determined according to a pure thresholding method (Table 1), a sepsis score that uses thresholds (Tables 2 and 3), or a sepsis index (Table 4). The method by which the sepsis state is obtained may be preset on the sepsis monitor 106 or may be selected by a user of the sepsis monitor 106. Stated another way, the sepsis monitor 106 may be capable of implementing all three methods of obtaining the patient's sepsis state and the user may select from one of the methods with input to the input device 120.

As illustrated in FIG. 17 when the pure thresholding method is selected, operation 1708 applies the set of rules (from operation 1612) by comparing the one or more physiological parameters to one or more thresholds associated with the one or more physiological parameters. Thereafter, operation 1712 determines the sepsis state for the patient based on the comparison (see Table 1, for example). The one or more thresholds may include a plurality of thresholds associated with possible sepsis states of the patient. Possible sepsis states may include a state of no sepsis, a state of potential onset of sepsis, a state of severe sepsis, and a state of septic shock. Additionally, the plurality of thresholds may include thresholds associated with nominal limits of the one or more physiological parameters, thresholds associated with abnormal limits of the one or more physiological parameters, and thresholds associated with critical limits of the one or more physiological parameters.

When the sepsis score is used to determine the patient's sepsis state, the method 1700 proceeds to operation 1716, which applies the set of rules by comparing the one or more physiological parameters to one or more baseline values for the one or more physiological parameters, where the one or more baseline values may be specific to the patient 104. Operation 1720 may determine a sepsis score based on the comparison while operation 1724 then determines the sepsis state of the patient 104 based on the sepsis score (see Tables 2 and 3 and FIG. 9). The baseline values may be determined in the same or similar manner as the thresholds listed in Table 1. In at least one embodiment, the method implemented in Table 1 may be used to generate a sepsis score as in Tables 2 and 3. In other words, the sepsis score may be affected by the conditions listed in Table 1.

When the sepsis index is selected in operation 1704, operation 1728 applies the set of rules by determining a difference between a first physiological parameter of the one or more physiological parameters measured at a first time and the first physiological parameter measured at a second time while operation 1732 includes determining an index value based on the difference. Thereafter, the sepsis state for the patient may be determined based on the index value in operation 1736. The method 1700 may determine the index value based on the difference by multiplying the difference by a coefficient associated with the first physiological parameter to generate an impact value, and determining the index value by adding the impact value to a base index value, where the base index value is determined by baseline values for the one or more physiological parameters specific to the patient (see Table 4 and FIG. 11). As discussed above, a value of the coefficient may be based on a reliability of the first physiological parameter in predicting the sepsis state. In at least one embodiment, the coefficient varies as the reliability of the first physiological parameter in predicting the sepsis state changes. As may be appreciated, operations 1728 and 1732 may be carried out for each physiological parameter being monitored so that the overall index value takes all parameters into account.

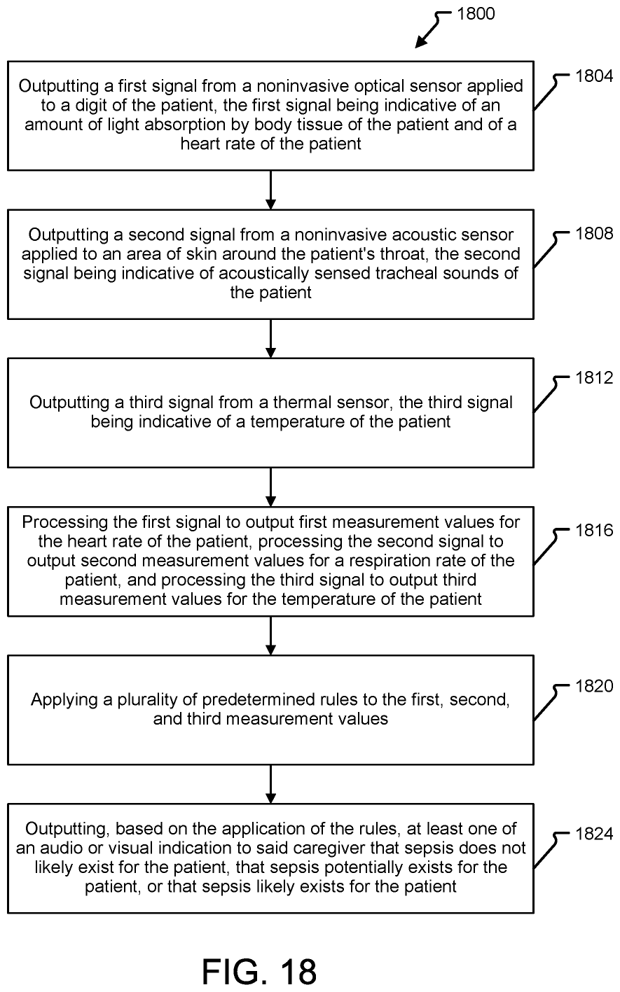

FIG. 18 illustrates a method 1800 that relates to electronically monitoring signals which are indicative of a patient condition to determine when to warn a caregiver of a condition which is indicative of sepsis in a patient 104.

Operation 1804 includes outputting a first signal from a noninvasive optical sensor applied to a digit of the patient 104. The first signal may be indicative of an amount of light absorption by body tissue of the patient 104 and/or of a heart rate of the patient 104. The first signal may be generated by a pulse oximeter or similar device.

Operation 1808 includes outputting a second signal from a noninvasive acoustic sensor applied to an area of skin around the patient's throat. The second signal may be indicative of acoustically sensed tracheal sounds of the patient 104 for the purpose of detecting respiration rate.

Operation 1812 includes outputting a third signal from a thermal sensor. The third signal may be indicative of a temperature of the patient 104.

Operation 1816 includes processing the first signal to output first measurement values for the heart rate of the patient 104, processing the second signal to output second measurement values for a respiration rate of the patient 104, and processing the third signal to output third measurement values for the temperature of the patient 104.

Operation 1820 includes applying a plurality of predetermined rules to the first, second, and third measurement values (see Tables 1-4 and related text and figures).

Operation 1824 includes outputting, based on the application of the rules, at least one of an audio or visual indication to the caregiver that sepsis does not likely exist for the patient 104, that sepsis potentially exists for the patient 104, or that sepsis likely exists for the patient 104.

FIGS. 16-18 have been discussed with reference to monitoring specific physiological parameters of a patient 104 for the purpose of determining a sepsis state. However, example embodiments are not limited the parameters mentioned above and additional or alternative parameters may be monitored. For example, in at least one embodiment, a sepsis monitor 106 utilizes Electroencephalograph (EEG) signals generated on from the scalp/cranium of a patient 104. EEG signals are run through various selections of pre-processing, metric analysis, and post-processing, as necessary, to extract features for classification of a patient's brain activity and neural function. Degradation in brain activity as quantified either by thresholds, characteristic definitions, or featured patterns (i.e., beta-wave patterns) may be indicative of Septic Encephalopathy and delirium, due to the onsetting symptoms of sepsis. Presence of Septic Encephalopathy gives the sepsis monitor 106, via post-processing into the sepsis index or sepsis score mentioned above, additional specificity for sepsis conditions and detection of onsetting sepsis in a patient 104.

In at least one embodiment, a sepsis monitor 106 utilizes blood analyte parameters such as Methemoglobin, Carboxyhemoglobin, Total Hemoglobin, Hematocrit, Deoxygenated Hemoglobin, Oxygenated Hemoglobin, Bilirubin, Oxygen Content (O₂ct) and Saturation Percent of Oxygen (SpO₂), perfusion index, for indications of sepsis state in a human. Using signals generated optically on from the skin surface of a human, monitoring the changes in blood analyte levels away from non-septic baseline values or beyond established thresholds indicate the onset of sepsis and septic status of a patient 104 by classifying physiological conditions, such as hypoperfusion. In the same fashion, as individual parameters or as the quantified state of hypoperfusion change to further indicate an increasing severity of septic state in a patient, the blood analyte parameters contribute to a sepsis index or sepsis score through pre-processing, metric analysis, and post-processing of the analytes, in the same or similar manner as that described above for other monitored parameters.

In addition to the typical form factor of a monitor in the professional clinical setting, an embodiment of a sepsis monitor 106 may exist as a wearable device that a patient temporarily interfaces with, where the wearable sepsis monitor 106 integrates the necessary electrical, optical, acoustic, motion, mechanical sensors, networking peripherals, and/or user interface components for sepsis detection and classification in a portable form factor that utilizes methods described herein for detecting sepsis through non-invasive monitoring of the patient. The unexpected results to having sepsis monitoring available outside of the clinical setting is highly preferential for the vast number of sepsis cases that present from the home environment. This portable sepsis monitor can communicate patient sepsis status and vital signs to clinical caretakers for remote monitoring applications.

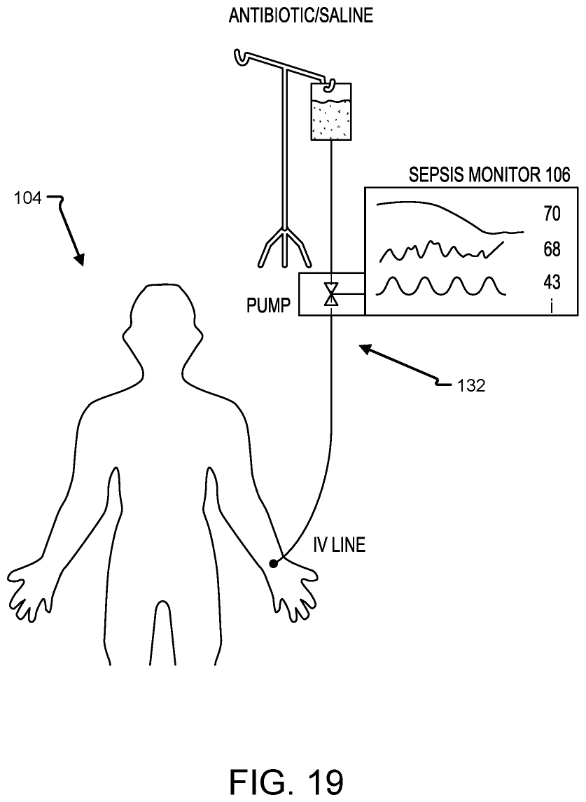

FIG. 19 illustrates an example of a sepsis monitor 106 and a sepsis treatment device 132 incorporated into a single unit. The treatment device 132 may comprise a saline/antibiotic/drug infusion pump or IV drip control. The sepsis monitor 106 and treatment device 132 may be incorporated within a single housing. Alternatively, the devices may be separately housed but proximately connected either physically (e.g., with wires) or wirelessly. For example, the treatment device 132 may be a “pluggable” device that is connectable to the sepsis monitor 106 through one or more interfaces.

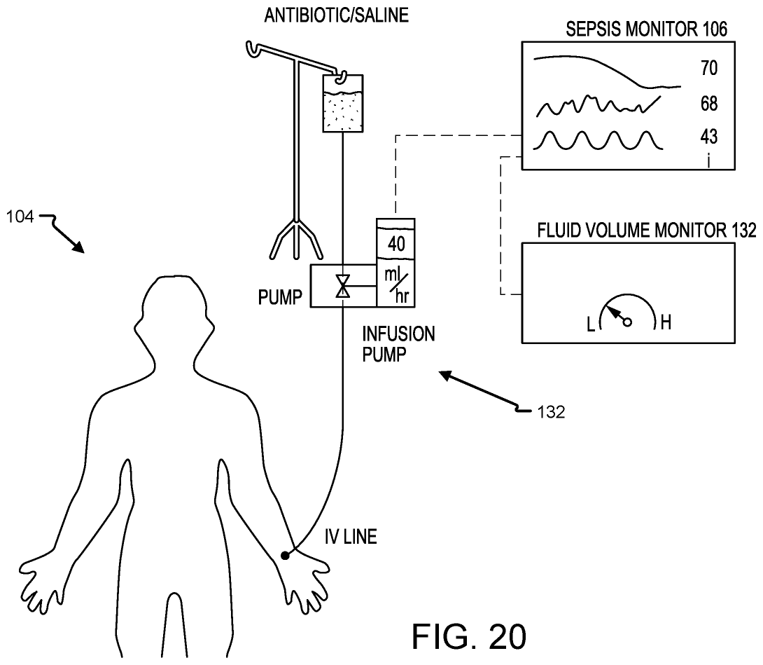

FIG. 20 illustrates an example of coordination between the sepsis monitor 106 and separate monitoring device(s) 132, such as a fluid volume monitor 132 to administer saline or other fluids. The fluid volume monitor 132 may have the same or similar structure and functionality as that set forth in U.S. Pat. No. 10,898,082, entitled “Method and Apparatus for Non-invasively Detecting Blood Volume Imbalances in a Mammalian Subject,” which is incorporated herein by reference. The fluid volume monitor 132 may monitor the amount of one or more fluids in a patient. In another example, the sepsis monitor 106 may interface with an arterial line blood pressure monitor to facilitate the administration of vasopressors for blood pressure control in a patient. This coordinated monitoring may implement one of the monitoring devices, or the administration device or other interface, as the feedback control authority for the administration of the treatment or therapy. The sepsis monitor 106 may act as the master in the cooperative configuration where the sepsis monitor 106 receives additional monitoring data from the external device, and the sepsis monitor 106 generate signals to control the administration of the treatment. The sepsis monitor 106 could also act as the subordinate device in the cooperative configuration to provide information and data on the sepsis state of the patient 104 while another device controls the administration of therapy.

A sepsis monitor has been disclosed in detail in connection with various embodiments. These embodiments are described by way of examples only and are not to limit the scope of the claims that follow. One of ordinary skill in art will appreciate many variations and modifications which can accomplish the same and similar objectives of purpose. For example, the values and physiological parameters used in Tables 1 to 4 may vary according to design preferences.

For purposes of explanation, numerous details are set forth in order to provide a thorough understanding of the present embodiments. It should be appreciated however that the techniques herein may be practiced in a variety of ways beyond the specific details set forth herein.

Furthermore, while the exemplary embodiments illustrated herein may show the various components of the system collocated, it is to be appreciated that the various components of the system can be located at distant portions of a distributed network, such as a communications network and/or the Internet, or within a dedicated secure, unsecured and/or encrypted system. Thus, it should be appreciated that the components of the system can be combined into one or more devices or collocated on a particular node/element(s) of a distributed network, such as a communications network. As will be appreciated from the description, and for reasons of computational efficiency, the components of the system can be arranged at any location within a distributed network without affecting the operation of the system.

Furthermore, it should be appreciated that the various links, including communications channel(s), connecting the elements (which may not be shown) can be wired or wireless links, or any combination thereof, or any other known or later developed element(s) that is/are capable of supplying and/or communicating data and/or signals to and from the connected elements. The term module as used herein can refer to any known or later developed hardware, software, firmware, or combination thereof that is capable of performing the functionality associated with that element. The terms determine, calculate and compute, and variations thereof, as used herein, are used interchangeably and include any type of methodology, process, mathematical operation, or technique.

While the above-described flowcharts/operational flows have been discussed in relation to a particular exemplary sequence of events, it should be appreciated that changes to this sequence can occur without materially affecting the operation of the embodiment(s). Additionally, the exact sequence of events need not occur as set forth in the exemplary embodiments, but rather the steps can be performed by one or the other device(s) in the system. Additionally, the exemplary techniques illustrated herein are not limited to the specifically illustrated embodiments but can also be utilized with the other exemplary embodiments and each described feature is individually and separately claimable.

As will be appreciated by one skilled in the art, aspects of the present disclosure may be embodied as a system, method, and/or computer program product. Thus, aspects of the present disclosure may be embodied entirely in hardware, entirely in software (including, but not limited to, firmware, program code, resident software, microcode), or in a combination of hardware and software. All such embodiments may generally be referred to herein as a circuit, a module, or a system. In addition, aspects of the present invention may be in the form of a computer program product embodied in one or more computer readable media having computer readable program code embodied thereon.

A computer readable medium as described herein may be a computer readable storage medium, examples of which include, but are not limited to, an electronic, magnetic, optical, electromagnetic, infrared, or semiconductor system, apparatus, or device, or any suitable combination thereof. As used herein, a computer readable storage medium may be any non-transitory, tangible medium that can contain or store a program for use by or in connection with an instruction execution system, apparatus, device, computer, computing system, computer system, or any programmable machine or device that inputs, processes, and outputs instructions, commands, or data. A non-exhaustive list of specific examples of a computer readable storage medium include an electrical connection to: a portable computer diskette, a floppy disk, a hard disk, a random access memory (RAM), a read-only memory (ROM), a Flash memory/storage interface (internal or external), a non-volatile RAM (NVRAM or NOVRAM), an erasable programmable read-only memory (EPROM or Flash memory), an electrically erasable programmable read-only memory (EEPROM), an optical fiber, a portable compact disc read-only memory (CD-ROM or DVD-ROM), an optical storage device, a magnetic storage device, or any suitable combination thereof. A computer readable storage medium can be any computer readable medium that is not a computer readable signal medium such as a propagated data signal with computer readable program code embodied therein.

Program code may be embodied as computer-readable instructions stored on or in a computer readable storage medium as, for example, source code, object code, interpretive code, executable code, or combinations thereof. Any standard or proprietary programming or interpretive language can be used to produce the computer-executable instructions. Examples of such languages include, but are not limited to: C, C++, C#, Dart, Python, R, React, Ruby, JAVA, JAVA Script, Swift, BASIC, Visual Basic, and Visual C++.

Transmission of program code embodied on a computer readable medium can occur using any appropriate medium including, but not limited to, wireless, wired, optical fiber cable, radio frequency (RF), or any suitable combination thereof.

The program code may execute entirely on a user's/operator's/administrator's computer, partly on such a computer, as a stand-alone software package, partly on the user's/operator's/administrator's computer and partly on a remote computer, or entirely on a remote computer or server. Any such remote computer may be connected to the user's/operator's/administrator's computer through any type of network, including a local area network (LAN) or a wide area network (WAN), or the connection may be made to an external computer (for example, through the Internet using an Internet Service Provider).

Additionally, the systems, methods and protocols described herein can be implemented to improve one or more of a special purpose computer, a programmed microprocessor or microcontroller and peripheral integrated circuit element(s), an ASIC or other integrated circuit, a digital signal processor, a hard-wired electronic or logic circuit such as discrete element circuit, a programmable or custom ASIC logic device such as PLD, PLA, FPGA, PAL/GAL, a smartphone, or any other comparable means. In general, any device capable of implementing a state machine that is in turn capable of implementing the methodology illustrated herein can benefit from the various communication methods, protocols, and techniques according to the disclosure provided herein.

Examples of the processors and/or microprocessors as described herein include, but are not limited to, at least one of Qualcomm® Snapdragon® family of processors, the Intel® Core™, Xeon®, Atom™, or Itanium® families of processors, the AMD® FX™ or Kaveri family of processors, Texas Instruments® Jacinto C6000™ automotive infotainment processors, Texas Instruments® OMAP™ automotive-grade mobile processors, ARM® Cortex™ processors, other industry-equivalent processors, and may perform computational functions using any known or future-developed standard, instruction sets, libraries, and/or architecture.

Furthermore, the disclosed methods may be readily implemented in software using object or object-oriented software development environments that provide portable source code that can be used on a variety of computer, workstation, or mobile device platforms, e.g., smartphones or mobile phones or vehicles. Alternatively, the disclosed system may be implemented partially in hardware using standard logic circuits or a VLSI design. Whether software or hardware is used to implement the systems in accordance with this invention is dependent on the speed and/or efficiency requirements of the system, the particular function, and the particular software or hardware systems or microprocessor or microcomputer systems being utilized. The methods illustrated herein however can be readily implemented in hardware and/or software using any known or later developed systems or structures, devices and/or software by those of ordinary skill in the applicable art from the functional description provided herein and with a general basic knowledge of the computer and image processing arts.

Moreover, the disclosed methods may be readily implemented in software executed on programmed general-purpose computer, a special purpose computer, mobile device, smartphone, a microprocessor, or the like. In these instances, the systems and methods of this invention can be implemented as a program embedded on personal computer or by means of a Common Gateway Interface (or similar), as a resource residing on a server or graphics workstation, as a routine embedded in a dedicated processing system, as a plug-in, or the like. The system can also be implemented by physically incorporating the system and method into a software and/or hardware system, such as the hardware and software systems of an image processor.

While this technology has been described in conjunction with numerous embodiments, it is evident that many alternatives, modifications, and variations would be, or are apparent, to those of ordinary skill in the applicable arts. Accordingly, it is intended to embrace all such alternatives, modifications, equivalents, and variations that are within the spirit and scope of this disclosure.

What is claimed is:

1. A system for monitoring sepsis, comprising:
one or more sensors that noninvasively monitor a patient to produce sensor data;
processing circuitry to:
derive, from the sensor data, one or more physiological parameters of the patient that are indicative of a sepsis state of the patient;
apply a set of rules to the one or more physiological parameters;
determine a sepsis state for the patient based on the set of rules applied to the one or more physiological parameters; and
output at least one signal indicative of the sepsis state of the patient; and
an output device that outputs a notification based on the at least one signal.

2. The system of claim 1, wherein the one or more sensors include a heart rate sensor, a temperature sensor, and a respiration rate sensor, and wherein the one or more physiological parameters of the patient include heart rate monitored by the heart rate sensor, temperature monitored by the temperature sensor, and respiration rate monitored by the respiration rate sensor.

3. The system of claim 1, wherein the output device includes a display that displays the notification to indicate the sepsis state of the patient.

4. The system of claim 1, wherein the processing circuitry applies the set of rules by comparing the one or more physiological parameters to one or more thresholds associated with the one or more physiological parameters, and wherein the processing circuitry determines the sepsis state for the patient based on the comparison.

5. The system of claim 4, wherein the one or more thresholds include a plurality of thresholds associated with possible sepsis states of the patient, wherein the possible sepsis states include a state of no sepsis, a state of potential onset of sepsis, a state of severe sepsis, and a state of septic shock.

6. The system of claim 5, wherein the plurality of thresholds include thresholds associated with nominal limits of the one or more physiological parameters, thresholds associated with abnormal limits of the one or more physiological parameters, and thresholds associated with critical limits of the one or more physiological parameters.

7. The system of claim 1, wherein the processing circuitry applies the set of rules by:
comparing the one or more physiological parameters to one or more baseline values for the one or more physiological parameters, the one or more baseline values being specific to the patient; and
determining a sepsis score based on the comparison, wherein the processing circuitry determines the sepsis state of the patient based on the sepsis score.

8. The system of claim 1, wherein the processing circuitry applies the set of rules by:
determining a difference between a first physiological parameter of the one or more physiological parameters measured at a first time and the first physiological parameter measured at a second time; and
determining an index value based on the difference, wherein the processing circuitry determines the sepsis state for the patient based on the index value.

8. The system of claim 7, wherein the processing circuitry determines the index value based on the difference by:
multiplying the difference by a coefficient associated with the first physiological parameter to generate an impact value; and
determining the index value by adding the impact value to a base index value, the base index value being determined by baseline values for the one or more physiological parameters specific to the patient.

9. The system of claim 8, wherein a value of the coefficient is based on a reliability of the first physiological parameter in predicting the sepsis state.

10. The system of claim 9, wherein coefficient varies as the reliability of the first physiological parameter in predicting the sepsis state changes.

11. A device for monitoring sepsis, comprising:
processing circuitry to:
derive, from sensor data of one or more noninvasive sensors, one or more physiological parameters of a patient that are indicative of a sepsis state of the patient;
apply a set of rules to the one or more physiological parameters;
determine a sepsis state for the patient based on the set of rules applied to the one or more physiological parameters; and
output at least one signal indicative of the sepsis state of the patient.

12. The device of claim 11, wherein the processing circuitry applies the set of rules by comparing the one or more physiological parameters to one or more thresholds associated with the one or more physiological parameters, and wherein the processing circuitry determines the sepsis state for the patient based on the comparison.

13. The device of claim 12, wherein the one or more thresholds include a plurality of thresholds associated with possible states of the sepsis state of the patient, wherein the possible states include a state of no sepsis, a state of potential onset of sepsis, a state of severe sepsis, and a state of septic shock.

14. The device of claim 13, wherein the plurality of thresholds include thresholds associated with nominal limits of the one or more physiological parameters, thresholds associated with abnormal limits of the one or more physiological parameters, and thresholds associated with critical limits of the one or more physiological parameters.

15. The device of claim 11, wherein the processing circuitry applies the set of rules by:
comparing the one or more physiological parameters to one or more baseline values for the one or more physiological parameters, the one or more baseline values being specific to the patient; and
determining a sepsis score based on the comparison, wherein the processing circuitry determines the sepsis state for the patient based on the sepsis score.

16. The device of claim 11, wherein the processing circuitry applies the set of rules by:
determining a difference between a first physiological parameter of the one or more physiological parameters measured at a first time and the first physiological parameter measured at a second time; and
determining an index value based on the difference, wherein the processing circuitry determines the sepsis state for the patient based on the index value.

17. The device of claim 16, wherein the processing circuitry determines the index value based on the difference by:
multiplying the difference by a coefficient associated with the first physiological parameter to generate an impact value; and
determining the index value by adding the impact value to a base index value, the base index value being determined by baseline values for the one or more physiological parameters specific to the patient.

18. The device of claim 17, wherein a value of the coefficient is based on a reliability of the first physiological parameter in predicting the sepsis state.

19. The device of claim 18, wherein coefficient varies as the reliability of the first physiological parameter in predicting the sepsis state changes.

20. A method of electronically monitoring signals which are indicative of a patient condition to determine when to warn a caregiver of a condition which is indicative of sepsis in a patient, the method comprising:
outputting a first signal from a noninvasive optical sensor applied to a digit of the patient, the first signal being indicative of an amount of light absorption by body tissue of the patient and of a heart rate of the patient;
outputting a second signal from a noninvasive acoustic sensor applied to an area of skin around the patient's throat, the second signal being indicative of acoustically sensed tracheal sounds of the patient;
outputting a third signal from a thermal sensor, the third signal being indicative of a temperature of the patient;
processing the first signal to output first measurement values for the heart rate of the patient, processing the second signal to output second measurement values for a respiration rate of the patient, and processing the third signal to output third measurement values for the temperature of the patient;
applying a plurality of predetermined rules to the first, second, and third measurement values; and
outputting, based on the application of the rules, at least one of an audio or visual indication to said caregiver that sepsis does not likely exist for the patient, that sepsis potentially exists for the patient, or that sepsis likely exists for the patient.
